(19) United States
(12) Patent Application Publication
(10) Pub. No.: US 2010/0021387 A1
(43) Pub. Date: Jan. 28, 2010

(54) Methods for Inducing Reversible Stasis

(75) Inventors: Mark B. Roth, Seattle, WA (US); Pamela A. Padilla, Denton, TX (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: FRED HUTCHINSON CANCER RESEARCH CENTER, Seattle, WA (US)

(21) Appl. No.: 12/478,677

(22) Filed: Jun. 4, 2009

Related U.S. Application Data
(63) Continuation of application No. 10/480,430, filed on Oct. 22, 2004, now abandoned.
(60) Provisional application No. 60/297,607, filed on Jun. 11, 2001.

(51) Int. Cl. A01N 1/02; C12N 5/00; A61K 49/00; A61K 31/4375; A61K 31/7088; C07D 491/12; A61P 35/04

(52) U.S. Cl. 424/9.2; 435/374; 435/2; 435/1.3; 514/279; 514/44 R; 546/37; 435/375

(57) ABSTRACT

The present invention concerns compositions and methods involving incubating biological materials under hypoxic or anoxic conditions to induce stasis or suspended animation. Methods of screening for compounds that induce stasis or compounds that increase the ability to undergo stasis are included. Such methods have ramifications for preserving biological materials as well as reducing or preventing trauma to biological materials. Also contemplated are methods for screening compounds that are active or more active under hypoxic conditions than normoxic conditions. Such methods can be used to identify antitumor compounds that would operate under hypoxic conditions in which tumor cells survive.

This application claims priority to U.S. Application Ser. No. 60/297,607 filed on Jun. 11, 2001, which is incorporated by reference in its entirety. The government may own rights in the present invention pursuant to grant number GM48435-05A1 from the National Institutes of Health.

The government may own rights in the present invention pursuant to grant number GM48435-05A1 from the National Institutes of Health.

### BACKGROUND OF THE INVENTION

1. Field of the Invention

The present invention relates generally to the fields of cell biology and physiology, as well as oncology. More particularly, it concerns methods and compositions involving exposing cells, tissues, or organisms to hypoxic or anoxic conditions. Compounds and methods for preserving and preventing damage to biological materials are specifically contemplated. Also contemplated are methods for screening compounds for the ability to induce stasis, or suspended animation, as well as for compounds with antitumor activity, and therapeutic compositions thereof.

2. Description of Related Art

Most animals are very sensitive to reduced levels of oxygen. Known vertebrate responses to low oxygen concentrations (hypoxia) include changes in carbohydrate metabolism, an increase in nitric oxide, and a stimulation of red blood cell and hemoglobin production (Guillemin et al., 1997). Hypoxia also can induce the expression of a select set of genes, including glycolytic enzymes, glycoprotein hormone erythropoietin and the inducible nitric oxide synthatase (Guillemin et al., 1997; Iyer et al, 1998). Hypoxia inducing factor (HI-1) has been shown to play a central role in this transcriptional response (Semenza, 1999-[α]; Semenza, 1999-[b]). Extreme hypoxia is central to the pathology of several diseases involving cardiac and pulmonary dysfunction (Semenza, 2000). Additionally, it is known that in certain solid tumors the cancerous cells that are hypoxic are more resistant to radiation and chemotherapy (Brown, 1999). Identification of the response organisms have to low oxygen tension may facilitate the development of treatment for rescue or prevention of damaged ischemic tissue, or for the destruction of tumor cells with low oxygen tensions.

Given its central role in physiology, several animal model systems have been developed to understand the response organisms have to reduced oxygen levels. The ability to survive anoxia (0% O₂) has been observed in small invertebrate organisms that lack a circulatory system and are therefore able to rapidly adapt to changes in oxygen levels (Foe et al, 1985; Hochachka et al., 1993). It has been shown that some invertebrates, such as Caenorhabditis elegans, Artemia franciscana, and Drosophila melanogaster, have the ability to survive in the absence of molecular oxygen (anoxia) (Anderson, 1978; Van Voorhies et al., 2000; Hand, 1993; Foe et al., 1985). The brine shrimp A. franciscana has been shown to survive four years of continuous anoxia and its response includes an arrest of development, a decrease in intracellular pH, a reduction in protein synthesis, and an accumulation of heat shock proteins (Hand, 1993; Clegg, 1997). It has been shown that both C. elegans and D. melanogaster can survive at least one day of anoxia exposure by arresting development until oxygen supply is reestablished (Van Voorhies et al., 2000; Foe et al., 1985). The survival of anoxia likely depends on the organisms ability to curb energy usage by shutting down nonessential cellular functions, maintain stable and low permeability of membranes, and the ability to synthesize ATP by glycolytic processes (Hochachka, 1986; Hochachka et al., 1996). Recent studies in D. melanogaster and mammalian tissue have demonstrated that the nitric oxide/cyclic GMP signaling pathway is involved in the response to oxygen deprivation (Wingrove et al., 1999; Clementi et al., 1999; Giulivi, 1998).

The ability to induce stasis (or suspended animation) in more developed organisms has not been previously demonstrated. This would provide ways of screening for stasis-inducing compounds that may have applicability to other vertebrate organisms, including mammals. Such applicability may extend to inducing stasis in cells, tissues, organs, systems, and entire organisms. Thus, the ability to suspend movement and/or development has ramifications with respect to short- or long-term preservation of biological material. In addition to the advantages of preservation by itself, preservation also may facilitate trauma or wound therapy, transportation of biological materials, as well as manipulation of biological materials.

Furthermore, because anoxic and hypoxic conditions simulate conditions under which a tumor in an animal subsists, antitumor compounds can be identified using organisms susceptible to stasis. While antitumor (anticancer) therapies exist, there is a continued need for new or improved methods of treating tumors.

The present invention demonstrates the ability to induce stasis in an organism and provides methods and compositions that address the needs identified above.

### SUMMARY OF THE INVENTION

The present invention takes advantage of the discovery that organisms, including vertebrate organisms can undergo stasis when incubated under anoxic or hypoxic conditions.

The present invention comprises methods of inducing stasis in biological materials—including organisms—as well as methods of modulating biological materials undergoing stasis or in stasis. As discussed herein, the invention extends to biological materials including cells—fertilized and unfertilized—tissues, organs, and parts of organisms, and entire organisms. It is specifically contemplated that methods and compositions with respect to one type of biological material may be implemented with respect to all other types of biological materials. In many instances, the organism is a vertebrate, while in others it is an invertebrate. Where the organism is invertebrate, embodiments include, but are not limited to Caenorabditis elegans or C. elegans. Vertebrate organisms include mammals, reptiles, amphibians, birds, and fish. Mammals are specifically contemplated, including those of veterinary, agricultural, and research importance, such as canine, feline, bovine, ovine, porcine, caprine, rodent, lagomorph, and swine. Humans, are specifically contemplated to be organisms for which the methods of the invention are applicable. Fish, including those of veterinary and aquacultural importance include, but are not limited to, Danio rerio, salmon, catfish, halibut, tuna, sea bass, red snapper, dover sole, petrale sole, tilapia, swordfish, mahi mahi, mackerel, yellowtail, skipper jack, opa, amberjack, barracuda, black drum, black grouper, cobia, flounder, gag grouper, jack crevalle, jewfish, king mackerel, ladyfish, lane snapper, mangrove snapper, mutton snapper, permit, pompano, redfish, red grouper, sheepshead, snook, spanish mackerel, spotted seatrout, tarpon, tripletail, yellowtail snapper, other bony fish, as well as cartilaginous fish such as sharks and rays, and shellfish. Birds used in embodiments of the invention include, but are not limited to, chickens, geese, ducks, pheasants, ostriches, emu, quails, and turkeys.

In some embodiments, stasis is induced in biological material by exposing or incubating the biological material under hypoxic or anoxic conditions sufficient to induce stasis of the biological material. It is contemplated that “sufficient to induce stasis” means that the material is exhibiting signs of stasis, i.e., for a finite length of time (as opposed to death) there is lack of movement, absence of cell division, reduction in cell division, absence of heartbeat, reduced heart beat, and lack of or reduction in developmental progression as observed by light microscopy.

As discussed in further detail below, hypoxic conditions include conditions in which the oxygen concentration is less than 20.8%—the concentration of normal atmospheric conditions—and as low as 0% (anoxic conditions); thus hypoxic conditions includes anoxic conditions unless otherwise specified; it is contemplated that hypoxic conditions with more than 0% oxygen are part of the invention. In some embodiments, oxygen concentration is less than 20, 19, 18, 17, 16, 15, 14, 13, 12, 11, 10, 9, 8, 7, 6, 5, 4, 3, 2, or 1%. In further embodiments, oxygen concentration is 0% or greater, or is between 0.5% and 20.8%. Methods implemented under anoxic conditions may also be implemented under hypoxic conditions and vice versa; both are contemplated as part of the present invention. It is also contemplated that in most embodiments of the invention, biological material will be restored to normoxic conditions, allowing for stasis to be reversed. It is further contemplated that minimal damage or harm to the biological material will result from being in stasis under the conditions described herein.

In all methods of the claimed invention, biological material may be exposed to temperatures lower than room temperature, including temperatures that will freeze the biological material, depending upon the liquid in or with which the biological material is incubated or perfused. Lowering of temperature may increase the duration that the biological material may undergo reversible stasis, preserve biological material, prevent damage or further damage to biological material, allow the biological material to undergo reversible stasis, increase the length of time biological material may be preserved, or increase the efficacy of a stasis inducer. In some embodiments, biological materials may be exposed to temperatures that allow the biological material to be frozen. For example, in some embodiments of the invention, sex cells or fertilized eggs are treated according to methods of the invention for use at a subsequent time. Alternatively, biological material may be incubated under hypoxic or anoxic conditions and placed in a temperature lower than room temperature either to prevent damage to biological material or to prevent further damage to biological material, such as to stave off the onset of trauma.

Thus, in further embodiments, the present invention includes methods for cryopreserving biological material comprising: first incubating the biological material under hypoxic or anoxic conditions for an effective amount of time for the biological material to enter stasis; and then cryopreserving the biological material. Cryopreserving biological material may involve steps generally used in cryopreservation, including steps of vitrification. Therefore, in further embodiments of the invention, steps of perfusing biological materials, particularly organs or tissues, with cryoprotectant agents are contemplated as part of the invention to protect the biological material.

In certain other embodiments of the present invention, methods are included for preserving biological material, particularly organ or tissues. Such methods include first incubating the biological material under hypoxic or anoxic conditions for an effective amount of time for the biological material to enter stasis and then lowering the temperature of the biological material.

In addition to hypoxic/anoxic conditions, another way of inducing stasis is to administer and effective amount of a stasis inducer compound, which is a compound capable of inducing a biological material to enter stasis, preferably reversible stasis.

An “effective amount” of a compound, generally, refers to an amount sufficient to detectably and repeatedly achieve a particular result. In the context of the present invention, one result sought is to induce stasis or suspended animation in a biological material. An effective amount of a stasis inducer, for example, would eliminate any detectable movement of the biological material, including, if appropriate, any detectable movement in the whole organism. More rigorous definitions may apply, including reduction or inhibition of cellular metabolism. Alternatively, in some embodiments the particular result desired is the treatment of a cancer, particularly a tumor. A “therapeutically effective amount” refers to any amount of a substance that promotes or enhances the well-being of the patient with respect to the medical treatment of his cancer. A list of nonexhaustive examples of this includes extension of the patient's life by any period of time; decrease or delay in the neoplastic development of the disease; decrease in hyperproliferation; reduction in tumor growth; delay of metastases; reduction in the proliferation rate of a cancer cell or tumor cell; induction of apoptosis in any treated cell or in any cell affected by a treated cell; and a decrease in pain to the patient that can be attributed to the patient's condition.

The present invention further concerns methods of screening for compounds that are candidates for cancer treatment. Such compounds may be antitumor compounds because of their ability to act under conditions of hypoxia, but not under conditions of normoxia. Alternatively, they may be stasis inducing compounds, that is, compounds that induce biological materials to undergo stasis. Furthermore, compounds that increase the efficacy of hypoxic conditions to induce stasis or that reduce any damage from stasis can be identified in screens of the present invention. The compounds to be screened include, but are not limited to small chemical molecules, peptides, polypeptides, nucleic acids, combinations and analogs thereof, which may be natural or synthetic products. Large-scale screening assays may be employed for screening methods of the invention. Libraries may be implemented, as well as high throughput analysis.

Methods of screening for an antitumor compound comprise: a) incubating a first anoxia- or hypoxia-resistant organism under hypoxic or anoxic conditions sufficient to permit the organism to enter stasis; b) incubating the first organism with a candidate compound; c) observing the first organism for viability; and d) comparing the first organism's viability against a second anoxia-resistant organism's viability incubated under normoxic conditions in the presence of the candidate compound. Viability of the second organism and lack of viability of the first organism identifies the candidate compound as an anti-tumor compound. It is specifically contemplated that any biological material may be implemented in this assay. “Anoxia-resistant” biological material (including anoxia-resistant organisms) have an ability to survive without oxygen without exhibiting harmful effects, which include, but are not limited to, developmental or physiological defects, such as brain damage, damage to the nervous system, or cardio-pulminary issues that result in tissue damage. “Hypoxia-resistant” biological material and organisms have an ability to survive under hypoxic conditions without exhibiting such harmful effects described above. The use of other biological materials, such as cells or tissues, is specifically contemplated for use with the present method of screening for antitumor compounds. In some embodiments, the candidate compounds are removed from the biological materials.

Evaluating viability may include evaluating the biological materials for movement, cell division, developmental progression, or other metabolic activities. Evaluation of gross changes such as heartbeat, cell division, movement, and developmental progression may be evaluated using an optical aid, such as a light microscope and camera. Metabolic activities, such as phosphorylation or ATP:ADP ratios, can be evaluated by methodology well known to those of skill in the art.

Compositions identified by the screening methods of the invention form part of the present invention. Thus, the invention includes an antitumor composition comprising an antitumor compound identified by a process comprising: a) incubating a first anoxia-resistant organism under hypoxic conditions sufficient to induce stasis; b) incubating the first organism with a candidate compound; and c) comparing the first organism's viability against a second anoxia-resistant organism's viability incubated under normoxic conditions in the presence of the candidate compound. As discussed earlier, in any embodiments of the invention, other biological material may be substituted for an organism. The compound is an antitumor compound If the first anoxia-resistant organism is no longer viable and the second anoxia-resistant organism is viable after incubation with the candidate compound. Any embodiment discussed above may be employed with this method and composition. A hypoxia-resistant organism may be employed with this method in place of an anoxic-resistant organism.

Chloromethyl-X-rosamine (CAS registry number: 167095-09-2 1H,5H,11H,15H-Xantheno[2,3,4-ij:5,6,7-i′j′]diquinolizin-18-ium, 9-[4-(chloromethyl)phenyl]-2,3,6,7,12,13,16,17-octahydro-, chloride; also known as (MITOTRACKER RED, Molecular Probes, Eugene Oreg.)) has been identified as compound that affects biological material under hypoxic conditions but not under normoxic conditions. This compound, or a derivative or analog thereof, may constitute an anti-cancer compound that can be employed as an anti-tumor compound for administration to a patient with a tumor.

Also included as part of the invention are methods for killing tumor cells in a patient with a tumor comprising administering to the patient a therapeutically effective amount of an antitumor compound identified by the process comprising:
a) incubating a first anoxia-resistant organism under hypoxic conditions sufficient to permit the organism to enter stasis;
b) incubating the first organism with a candidate compound;
c) observing the first organism for viability; and
d) comparing the first organism's viability against a second anoxia-resistant organism's viability incubated under normoxic conditions in the presence of the candidate compound,
   wherein viability of the second organism and lack of viability of the first organism identifies the candidate compound as an anti-tumor compound.

The method, in some embodiments, further comprises performing surgery on the patient or administering at least one other anti-cancer treatment, such as chemotherapy, radiotherapy, immunotherapy or gene therapy.

The compound identified above as chloromethyl-X-rosamine (MITOTRACKER RED) is specifically contemplated for use in the present invention.

Other screening methods include screening for a compound that induces stasis in biological material comprising: a) incubating a first biological material capable of undergoing stasis with a candidate compound; and b) evaluating the first biological material for evidence of stasis. The compound is a stasis inducer if the first biological material exhibits evidence of stasis after exposure to the compound. In some embodiments, the method further comprises c) comparing the ability to induce stasis in the first biological material with a second biological material not incubated or no longer incubated with the candidate compound. In still further embodiments, it also comprises d) removing the compound from the first biological material; and e) evaluating the first biological material for loss of stasis. The compound is a reversible stasis inducer if the first organism exhibits stasis after incubation with the compound, but no longer exhibits stasis after the compound is removed. In some embodiments, a reversible stasis inducer can be identified by comparing biological material that was exposed to the candidate compound and the same biological material but in the absence of the candidate compound or after the candidate compound has been removed.

In still further embodiments, the present invention concerns methods of screening for a compound that improves the ability of biological material to survive anoxia or hypoxia or to undergo stasis comprising: a) incubating a first biological material capable of undergoing stasis under hypoxic or anoxic conditions; b) exposing the first biological material to a candidate compound; c) incubating a second biological material capable of undergoing stasis under the same hypoxic conditions as the first biological material; d) comparing the first biological material and the second biological material. A candidate compound is one that improves the ability of a biological material to survive under anoxic or hypoxic conditions. Any of the embodiments described herein may be applied to practice any of the screening methods of the invention. The invention includes the use of biological material that is capable of undergoing stasis during a particular point in its development or lifetime, but is not capable of undergoing stasis at the time of testing.

Stasis inducer compounds, including reversible stasis inducer compounds (compounds that induce reversible stasis) identified by screening methods form part of the present invention.

Methods of the invention include using the identified compounds. Thus, the present invention includes methods of inducing stasis in biological material by administering to the biological material an effective amount of a stasis inducer compound identified by processes described herein. It also includes methods of treating a tumor or inhibiting its growth using the antitumor compounds of the claimed invention. Such compounds may be formulated in pharmaceutically acceptable formulations and administered to a tumor cell or to a patient using routine routes of administration.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The present invention is based on the discovery that both invertebrate and vertebrate organisms can be induced to undergo temporary stasis and yet subsequently achieve normal development or have normal function reestablished. Methods and compositions taking advantage of this are presented herein.

### I. Stasis or Suspended Animation

In “stasis” or “suspended animation,” a cell, tissue, or organism (collectively referred to as “biological material”) is living, but cellular functions necessary for cell division, developmental progression, metabolic state are slowed or even stopped. This state is desirable in a number of contexts. Stasis can be used as a method of preservation by itself, or it may be induced as part of a cryopreservation regimen. Biological materials may be preserved for research use, for transportation, for transplantation, for therapeutic treatment (such as ex vivo therapy), and to prevent the onset of trauma, for example. Stasis with respect to entire organisms have similar uses. For instance, transportation of organisms could be facilitated if they had entered stasis. This might reduce physical and physiological damage to the organism by reducing or eliminating stress or physical injury. Biological material contemplated for use with the present invention include material derived from invertebrates and vertebrates, including mammals; biological materials includes organisms. In addition to humans, the invention can be employed with respect to mammals of veterinary or agricultural importance including those from the following classes: canine, feline, equine, bovine, ovine, murine, porcine, caprine, rodent, lagomorph, lupine, and ursine. The invention also extends to fish and birds. Sex cells, somatic cells, fertilized eggs, embryos, and fetuses fall within the term “biological materials.”

“Hypoxia” occurs when the normal physiologic levels of oxygen are not supplied to a cell or tissue. “Normoxia” refers to normal physiologic levels of oxygen for the particular cell type, cell state or tissue in question. “Anoxia” is the absence of oxygen. “Hypoxic conditions” are those leading to cellular hypoxia. These conditions depend on cell type, and on the specific architecture or position of a cell within a tissue or organ, as well as the metabolic status of the cell.

For purposes of the present invention, hypoxic conditions include conditions in which oxygen concentration is at or less than normal atmospheric conditions, that is less that 20.8, 20, 19, 18, 17, 16, 15, 14, 13, 12, 11, 10, 9, 8, 7, 6, 5, 4, 3, 2, 1, 0.5, 0%. An oxygen concentration of zero percent defines anoxic conditions. Thus, hypoxic conditions include anoxic conditions, although in some embodiments, hypoxic conditions of not less than 0.5% are implemented. As used herein, “normoxic conditions” constitute oxygen concentrations of around 20.8% or higher.

Standard methods of achieving hypoxia or anoxia are well established and include using environmental chambers that rely on chemical catalysts to remove oxygen from the chamber Such chambers are available commercially from, for example, BD Diagnostic Systems (Sparks, Md.) as GASPAK Disposable Hydrogen+Carbon Dioxide Envelopes or BIO-BAG Environmental Chambers. Alternatively, oxygen may be depleted by exchanging the air in a chamber with a non-oxygen gas, such as nitrogen. Oxygen concentration may be determined, for example using a FYRITE Oxygen Analzyzer (Bacharach, Pittsburgh Pa.).

A. Preservation

The present invention can be used for cryopreservation (preservation at very low temperatures) and vitrification (solidification without freezing). As discussed in U.S. Pat. Nos. 5,952,168, 5,217,860, 4,559,258 and 6,187,529 (incorporated specifically by reference), biological materials can be preserved, for example, for keeping transplantable or replaceable organs long-term.

In this context, biological materials are first induced to enter stasis. Within certain embodiments of the invention, biological materials are first incubated under anoxic or hypoxic conditions to induce stasis. In some embodiments the biological materials are first induced to enter stasis prior to cryopreservation or vitrification. It is contemplated that biological materials may be kept under hypoxic conditions for more than 5, 10, 15, 20, 25, 30, 35, 40, 45, 50, 55 minutes, 1, 2, 3, 4, 5, 6, 7, 8, 9, 10, 11, 12, or more hours, 1, 2, 3, 4, 5, 6, 7 or more days prior to cryopreservation or vitrification. Although in some embodiments the material will not be kept under hypoxic conditions for more than 3 days prior to cryopreservation or vitrification.

In certain other embodiments of the present invention, methods for preserving biological materials, particularly organs or tissues, comprise: first incubating the biological material under hypoxic or anoxic conditions for an effective amount of time for the biological material to enter stasis; and lowering the temperature of the biological material. Within this embodiment, the temperature of the biological material is lowered to below 37.5° C. but above approximately 10° C.

In other embodiments biological materials are incubated under hypoxic or anoxic conditions and the materials are frozen or infused with cryoprotectant agents before stasis is achieved. Various methods of cryopreservation are described in patents cited above, which are specifically incorporated by reference. To implement cryopreservation processes, one method calls for cooling the material, perfusing it with a cryoprotectant agent, often containing glycol ethers, and then perfusing with an inert fluid to replace the cryoprotectant agent, and finally cooling the material even further. The cryoprotectant agent protects biological materials when temperatures are lowered by interacting with water to prevent the ordering of water molecules (freezing) at low temperatures. Inert fluids that can be employed are any liquids that remain so at low temperatures with low viscosity and low toxicity to biological materials. In the context of the present invention, temperatures of lower than room temperature are specifically preferred, including temperatures around −196° C. (−321° F.) and in the following ranges: about −196° C. to about 0° C. or about −100° C. to about −50° C.

With vitrification, generally, the lowest temperature to which a solution can possibly supercool without freezing is the homogeneous nucleation temperature at which temperature ice crystals nucleate and grow, and a crystalline solid is formed from the solution. Vitrification solutions have a glass transition temperature at which temperature the solute vitrifies or becomes a non-crystalline solid. Because of the kinetics of nucleation and crystal growth, it is effectively impossible for water molecules to align for crystal formation at temperatures much below the glass transition temperature. In addition, on cooling most dilute aqueous solutions to the glass transition temperature, a homogeneous nucleation temperature is encountered before the glass transition temperature, and ice nucleation occurs, making vitrification of the solution not possible. To make such solutions useful in the preservation of biological materials by vitrification, it is therefore necessary to change the properties of the solution so that vitrification occurs instead of ice crystal nucleation and growth. Such cryoprotectants and regimens are described in U.S. Pat. No. 6,194,137, which is specifically incorporated by reference.

B. Preventing Trauma

In certain embodiments, the present invention may find use in the treatment of patients undergoing, or are susceptible to trauma. Trauma sets of a series of biochemical processes, such as clotting, inflammation, hypotension, and may ultimately lead to shock. While these processes are designed to defend the body against traumatic insult, they may prove harmful and, in some instances, may be fatal. Trauma may result from external causes that result in an acute reduction in circulation such as gunshot wounds, surgical trauma, acute reduction in circulation due to stroke or heart attack, or reductions in circulation due to non-invasive stress, such as exposure to cold or radiation.

Therefore, the present invention contemplates the placement of organs, limbs and even whole organisms into stasis as a way of protecting them from the detrimental effects of trauma. In a specific scenario, where medical attention is not readily available, induction of stasis in vivo or ex vivo can “buy time” for the subject, either by bringing medical attention to the subject, or by transporting the subject to the medical attention.

### II. Methods of Screening for Stasis-Inducers and Anti-Tumor Compounds

Screening methods are contemplated by the present invention. Compounds can be screened for an ability to induce stasis in a cell, tissue, or organism. In some embodiments, organisms known to be capable of undergoing stasis are employed. Thus, nematodes, zebrafish, or fruit flies may be used to evaluate whether a candidate compound can induce stasis. Alternatively, cells, tissues, or organisms not yet known to be capable of undergoing stasis are employed.

Also, because tumor cells can survive in hypoxic conditions, compounds can be screened for an ability to induce a physiological effect under conditions of hypoxia but not under conditions of normoxia. Consequently, such compounds will not have any effect on biological materials that are not under hypoxic conditions. Candidate antitumor compounds will exhibit differential activity with respect to oxygen concentrations. Compounds that are able to act on biological material only under hypoxic conditions and not under normoxic conditions may have other uses as well. For example, such a compound may be used in the preservation of biological materials either short-term or long-term. It may reduce physiological damage to cells/tissues/organisms that are incubated or kept under hypoxic conditions. A compound that acts under differential oxygen conditions, “differential oxygen compound,” may also have uses in preventing aging or senescence.

The present invention also includes methods of screening for compounds whose efficacy is increased under conditions of hypoxia or anoxia compared to efficacy under normoxic conditions. With such compounds, biological material can be incubated under hypoxic conditions and then the compound can be administered. The methods of screening described herein can be employed to identify compounds that kill cells under hypoxic or anoxic conditions.

The present invention further comprises methods for identifying modulators of the hypoxic/anoxic stasis pathway (pathway that contributes to induction of stasis under hypoxic or anoxic conditions), as well as modulators of biological material already in stasis. Thus, it is contemplated that such modulators are substances that affect the ability of biological material to enter stasis as well as the ability of biological material to be maintained in stasis and exit stasis (no longer be in stasis). A modulator is one that has any effect on these processes. These assays may comprise random screening of large libraries of candidate substances; alternatively, the assays may be used to focus on particular classes of compounds selected with an eye towards structural attributes that are believed to make them more likely to modulate the function of gene products in the hypoxic/anoxic stasis pathway.

By function, it is meant that one may assay for a measurable effect on the ability to induce or to modulate stasis in a cell, tissue, or organism. To identify a hypoxic/anoxic stasis pathway modulator, one generally will determine the activity or level of stasis induction in the presence and absence of the modulator, wherein a modulator is defined as any substance that alters these characteristics. For example, a method generally comprises:
(a) admixing a candidate modulator with a biological material capable of undergoing stasis;
(b) subjecting the candidate modulator treated biological material to hypoxic/anoxic conditions for a time sufficient to induce stasis;
(c) measuring a characteristic associated with the entrance of the biological material into stasis; and
(d) comparing the characteristic measured in step (b) with the characteristic of a biological material untreated with the candidate modulator and under hypoxic/anoxic conditions,
wherein a difference between the measured characteristics indicates that said modulator is, indeed, a modulator of the stasis pathway.

Within certain embodiments of the invention more than one characteristic is measured. Suitable characteristics for measurement include but are not limited to time to entrance of stasis, time to exit from stasis, duration of stasis, biological parameters associated with stasis including movement, cell division, developmental progression, evaluation of gross changes such as heartbeat, cell division, and metabolic activities, such as phosphorylation and ATP:ADP ratios. Additional steps of the method can include a step of removing the candidate modulator from the biological materials held in stasis under hypoxic/anoxic conditions prior to measurement of characteristics in step (c) or removal of the biological material from the modulator and from the hypoxic/anoxic conditions prior to measurement of characteristics in step (c).

To identify a stasis pathway modulator that permits survival of a biological material that would otherwise perish under hypoxic/anoxic conditions, one generally will determine the activity or level of stasis induction in the presence and absence of oxygen, wherein a modulator is defined as any substance that alters these characteristics. For example, a method generally comprises:
(a) admixing a candidate modulator with a biological material not presently capable of undergoing stasis;
(b) incubating the candidate modulator admixed with the biological material under hypoxic or anoxic conditions for a time sufficient to induce stasis;
(c) determining whether the biological material survives the anoxic or hypoxic condition,
wherein survival of the biological material identifies the candidate modulator as a modulator of stasis.

Assays may be conducted in cell free systems, in isolated cells, or in organisms including transgenic animals, or they may be conducted using preparations from the biological material, such as isolated or purified mitochondria. In vitro assays may be employed, for example, to measure oxidative phosphorylation.

Methods of screening for modulators also include a method of screening for a modulator that affects the duration in or exit from stasis. Such a method may include the following:
(a) admixing a candidate modulator with biological material in stasis;
(b) determining whether stasis has been affected in the biological material;
(c) comparing the characteristic measured in step (b) with the characteristic of the biological material in the absence of the candidate modulator,
wherein a difference between the measured characteristics indicates that said candidate modulator is, indeed, a modulator of stasis in the biological material.

The present invention also comprises methods for identifying stasis inducers, preferably reversible stasis inducers that mimic the stasis induced by the hypoxic/anoxic stasis pathway (pathway that contributes to induction of stasis under hypoxic/anoxic conditions). Thus, it is contemplated that such stasis inducers are substances that permit a biological material to enter stasis and preferably to exit stasis when the substance is removed from the biological material. In general, such assays will permit the random screening of large libraries of candidate substances. Within one example, a method generally comprises:
(a) admixing a candidate compound with a biological material capable of undergoing stasis;
(b) determining whether the biological material enters stasis.
   Within certain embodiments, this method may also include a step in which a second biological material capable of undergoing stasis is subjected to hypoxic/anoxic conditions for a time sufficient to induce stasis and an additional step of comparing the first biological material and candidate compound and the second biological material that is in stasis such that suitable candidate compounds are those that mimic the hypoxic/anoxic induced stasis. Within yet another embodiment of this method, an additional step is included in which the biological material that has been successfully induced to undergo stasis is removed from the candidate compound to determine if the biological material is capable of exiting stasis.

It will, of course, be understood that all the screening methods of the present invention are useful in themselves notwithstanding the fact that effective candidates may not be identified. The invention provides methods for screening for such candidates, not solely methods of finding them.

As used herein the term “candidate substance” refers to any molecule that may potentially affect the ability of biological material to undergo stasis. The candidate substance may be a protein or fragment thereof, a small molecule, or even a nucleic acid molecule.

One may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are well known to those of skill in the art. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on genes and proteins in the hypoxic stasis pathway. Regardless of the type of inhibitor or activator identified by the present screening methods, the effect of the inhibition or activator by such a compound results in alteration in hypoxic stasis pathway activity as compared to that observed in the absence of the added candidate substance.

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

A technique for high throughput screening of compounds is described in WO 84/03564. Large numbers of small peptide test compounds are synthesized on a solid substrate, such as plastic pins or some other surface. Bound polypeptide is detected by various methods.

Compounds to be screened may be small molecules, peptides, peptide analogs, peptide mimetics, etc. Candidate compounds to be screened are not limited in any way, however, such compounds will be more promising if they are not harmful or caustic to biological materials. To screen a large number of compounds, libraries, high throughput assays, and arrays are contemplated to be of use for practicing the invention.

A. Chemical Libraries

The present invention involves, in some embodiments, screening many compounds for the ability to effect stasis of cells, tissues, or organisms. Alternatively, because tumor cells often frown under hypoxic conditions, screens for novel antitumor drugs can be discovered using the hypoxic conditions and biological materials described herein. Libraries of chemical compounds may be of any origin. For example, they may be small molecule chemical libraries or combinatorial chemical libraries, including peptide libraries.

Combinatorial chemical libraries can be used for the identification of novel lead compounds or for the optimization of a promising lead candidate that are pharmacologically active compounds. By pharmacologically active is meant that a compound may affect the functioning of a physiological process and have emerged as a promising and potentially powerful method for the acceleration of the drug discovery process. (Terrett, et al., 1995; Gallop, et al., 1994; Janda, 1994; Pavia, 1993).

A “combinatorial library” refers to a collection of compounds in which the compounds comprising the collection are composed of one or more types of subunits, such as natural or unnatural moieties, including nucleophilic compounds, acylating agents, aromatic compounds, heterocyclic compounds, ethers, amines, carboxylic acids, amides, esters, thioesters, compounds containing a carbon-hetero multiple bond, L-amino acids, D-amino acids, synthetic amino acids, nucleotides, sugars, lipids, carbohydrates. Alternatively, a “combinatorial library” may refer to a collection or set of “core molecules,” which vary as to the number, type or position of R or functional groups they contain and/or identity of molecules composing the core molecule. Examples of how to construct and implement combinatorial libraries can be found in U.S. Pat. Nos. 6,185,506; 6,184,389; 6,168,912; 6,153,375, each incorporated by reference.

Peptide or oligonucleotide libraries and related oligomeric structures can be employed as combinatorial libraries for screening. (See Gallop, supra, Geysen, et al., 1984; Lam, et al., 1991; Houghten, et al., 1991; Salmon, et al., 1993; Owens, et al., 1991; Bock., et al., 1992; Scott, 1990; Cwirla, et al., 1990; Devlin, et al., 1990; Simon, et al., 1992; Zuckermann, et al., 1992; Miller, et al., 1994; Zuckerman, et al, 1994; Terrett, et al., 1995; Cho, et al., 1993; Winkler et al, WO93/09668 (PCT/US92/10183)); Ostresh, et al., 1994.

Conventional small molecule libraries may also be used to screen compounds in the context of the present methods. (See. e.g., Simon, et al., 1992; Zuckermann, et al., 1992; Miller, et al., 1994; Zuckerman, et al, 1994; Terrett, et al., 1995; Cho, et al., 1993; Winkler et al, WO93/09668 (PCT/US92/10183)); Ostresh, et al., 1994; Bunin, et al., 1992; Bunin, et al., 1994; Virgilio, 1994; Kick, 1995; DeWitt, et al., 1993; Chen, et al., 1994; Beebe, et al., 1992; Moon, et al., 1994; Kurth, et al., 1994; Gordon, 1995; Patek, et al., 1994; Patek, et al., 1995; Campbell, et al., 1995; Forman, 1995; Rano, 1995; Dankwardt, et al., 1995; Deprez, et al., 1995; Ellman, U.S. Pat. No. 5,288,514).

Solid phase synthesis has been adapted from solid phase synthesis of peptides and oligonucleotides for use in the synthesis of small chemical libraries. Methods of synthesizing diverse chemical libraries on solid supports include split or mixed synthesis (Furka, et al., 1988; Furka, et al., 1991; Houghten, 1985; Erb, et al., 1994), encoded synthesis (Brenner, 1992; Nielsen, et al., 1993; Needels, et al., 1993; Nikolaiev, et al., 1993; Kerr, et al., 1993; Ohlmeyer, et al., 1993; Nestler, et al., 1994; Baldwin, et al., 1995), indexed synthesis (Pirrung, 1995; Smith, et al., 1994), or parallel and spatially addressed synthesis on pins (Geysen, et al., 1984; DeWitt, et al., 1993), beads (Merrifield, 1963), chips (Fodor, et al., 1991), and other solid supports (Atherton, 1989; Grubler, et al., 1994; Englebretsen, 1992; Frank, 1993; Frank, 1988; Schmidt. et al., 1993; Eichler, et al., 1991).

In some embodiments of the invention, plates with multiple wells may be used to screen large numbers of compounds. Compounds will then be evaluated for the ability to induce stasis or the ability to effect cell death in an organism under hypoxic or anoxic conditions.

B. Chip Technologies

Specifically contemplated by the present inventors are chip-based DNA technologies such as those described by Hacia et al. (1996) and Shoemaker et al (1996). Also included are protein-based chip technologies. Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating gene expression profiles of cells in stasis as compared to those not in stasis or in identifying genes involved in the stasis or oxygen sensor pathway.

### III. Identified Antitumor Compounds and Combination Treatments

As discussed above, the present invention can be implemented to identify antitumor compounds that would exert an antiproliferative effect on tumor cells but not normal cells. This is accomplished by assaying a compound in the presence and absence of oxygen and is termed “hypoxia screening method,” which means the screen is conducted at some point, under hypoxic or anoxic conditions. Compounds with potential antitumor activity identified in the hypoxia screening method are termed “hypoxic antitumor compounds.” A compound identified under conditions of anoxia could also be termed an “anoxic antitumor compound.” Among other things, the compounds could reduce tumor size, reduce tumor cell growth, induce apoptosis in tumor cells, reduce tumor vasculature, reduce or prevent metastasis, reduce tumor growth rate, accelerate tumor cell death, and kill tumor cells. In some embodiments the antitumor compound can be administered to a patient as part of an anticancer regimen that included other anticancer treatments in order to increase the effectiveness of a treatment with the compositions identified by the present invention. While the present invention is directed at antitumor compounds because of the conditions under which tumors may exist (hypoxic conditions), the compounds may be more generally applied with respect to cancer.

Chloromethyl-X-rosamine (CAS registry number: 167095-09-2 1H,5H,15H,15′-Xantheno[2,3,4-ij:5,6,7-i′j′]diquinolizin-18-ium, 9-[4-(chloromethyl)phenyl]-2,3,6,7,12,13,16,17-octahydro-, chloride; also known as (MITOTRACKER RED, Molecular Probes, Eugene Oreg.) has been identified as compound that affects biological material under hypoxic conditions but not under normoxic conditions. This compound, or a derivative or analog thereof, may constitute an anti-cancer compound that can be employed as an anti-tumor compound for administration to a patient with a tumor.

Examples of cancer contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, bladder cancer and any other cancer involving tumors—solid or liquid.

It may be desirable to combine the compositions of the present invention with other agents (secondary agents) effective in the treatment of hyperproliferative disease, such as anti-cancer agents, or with surgery. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cancer or tumor cells. This process may involve contacting the cells with a composition of the invention and the agent(s) or multiple factor(s) at the same time. This may be achieved by contacting the cell with a single composition or pharmacological formulation that includes both agents, or by contacting the cell with two distinct compositions or formulations, at the same time, wherein one composition includes a composition of the present invention and the other includes the second agent(s).

To address tumor cell resistance to chemotherapy and radiotherapy agents, a major problem in clinical oncology, gene therapy may be combined with treatment with the compositions of the present invention. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that hypoxic antitumor compound may be used similarly in conjunction with chemotherapeutic, radiotherapeutic, immunotherapeutic or other biological intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed; the hypoxic antitumor compound identified by the screening method of the claimed invention (“hypoxic screening method”) is “A” and the secondary anti-cancer agent, such as radio- or chemotherapy, is

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic compounds of the present invention to a patient will follow general protocols for the administration of chemotherapeutics, taking into account the toxicity, if any, of the compound. It is expected that the treatment cycles would be repeated as necessary. It also is contemplated that various standard therapies, as well as surgical intervention, may be applied in combination with the described anti-cancer therapy.

1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melpbalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a composition of the invention (for example, a hypoxic antitumor compound) or a chemotherapeutic or radiotherapeutic agent is delivered to a target cell or are placed in direct juxtaposition with the target cell. In combination therapy, to achieve cell killing or stasis, both agents may be delivered to a cell in a combined amount effective to kill the cell or prevent it from dividing.

3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy could also be used as part of a combined therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as mda-7 has been shown to enhance anti-tumor effects (Ju et al., 2000).

As discussed earlier, examples of immunotherapies currently under investigation or in use are immune adjuvants (e.g., Mycobacterium bovis, Plasmodium falciparum, dinitrochlorobenzene and aromatic compounds) (U.S. Pat. No. 5,801,005; U.S. Pat. No. 5,739,169; Hui and Hashimoto, 1998; Christodoulides et al., 1998), cytokine therapy (e.g., interferons α, β and γ; IL-1, GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. No. 5,830,880 and U.S. Pat. No. 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the anti-tumor therapies described herein.

i. Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers as described by Bajorin et al. (1988). The development of human monoclonal antibodies is described in further detail elsewhere in the specification.

ii. Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

iii. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

d. Genes

In yet another embodiment, the secondary treatment is a gene therapy in which a therapeutic polynucleotide (or second therapeutic polynucleotide if an antitumor compound is provided to a cell by providing a nucleic acid encoding the modulator) is administered before, after, or at the same time as an anti-tumor compound is administered. Delivery of an antitumor compound in conjunction with a vector encoding one of the following gene products will have a combined anti-hyperproliferative effect on target tissues. A variety of proteins are encompassed within the invention, some of which are described below. Table I lists various genes that may be targeted for gene therapy of some form in combination with the present invention.

i. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g. Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

ii. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano S et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

iii. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

e. Surgery

Approximately 60% of persons with cancer will undergo surgery of some type, which includes preventative, diagnostic or staging, curative and palliative surgery. Curative surgery is a cancer treatment that may be used in conjunction with the hypoxic antitumor compounds of the present invention and may be used in conduction with other therapies, such as chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

f. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al., 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Studies from a number of investigators have demonstrated that tumor cells that are resistant to TRAIL can be sensitized by subtoxic concentrations of drugs/cytokines and the sensitized tumor cells are significantly killed by TRAIL. (Bonavida et al., 1999; Bonavida et al., 2000; Gliniak et al., 1999; Keane et al., 1999). Ad-mda7 treatment of cancer cells results in the up-regulation of mRNA for TRAIL and TRAIL receptors. Therefore, administration of the combination of Ad-mda7 with recombinant TRAIL can be used as a treatment to provide enhanced anti-tumor activity. Furthermore, the combination of chemotherapeutics, such as CPT-11 or doxorubicin, with TRAIL also lead to enhanced anti-tumor activity and an increase in apoptosis. The combination of Ad-mda7 with chemotherapeutics and radiation therapy, including DNA damaging agents, will also provide enhanced anti-tumor effects. Some of these effects may be mediated via up-regulation of TRAIL or cognate receptors, whereas others may not. For example, enhanced anti-tumor activity with the combinations of Ad-mda7 and tamoxifen or doxorubicin (adriamycin) has been observed. Neither tamoxifen nor adriamycin are known to up-regulate TRAIL or cognate receptors.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

TABLE 1
Oncogenes

| Gene | Source | Human Disease | Function |
|---|---|---|---|
| Growth Factors | | | |
| HST/KS | Transfection | | FGF family member |
| INT-2 | MMTV promoter | | FGF family member |
| | Insertion | | |
| INTI/WNTI | MMTV promoter | | Factor-like |
| | Insertion | | |
| SIS | Simian sarcoma virus | | PDGF B |
| Receptor Tyrosine | | | |
| Kinases | | | |
| ERBB/HER | Avian erythroblastosis | Amplified, deleted | EGF/TGF-α/ |
| | virus; ALV promoter | Squamous cell | Amphiregulin/ |
| | insertion; amplified | Cancer; glioblastoma | Hetacellulin |
| | human tumors | | receptor |
| ERBB-2/NEU/HER-2 | Transfected from rat | Amplified breast, | Regulated by NDF/ |
| | Glioblastomas | Ovarian, gastric | Heregulin and EGF- |
| | | cancers | Related factors |
| FMS | SM feline sarcoma virus | | CSF-1 receptor |
| KIT | HZ feline sarcoma virus | | MGF/Steel receptor |
| | | | Hematopoieis |
| TRK | Transfection from | | NGF (nerve growth |
| | human colon cancer | | Factor) receptor |
| MET | Transfection from | | Scatter factor/HGF |
| | human osteosarcoma | | Receptor |
| RET | Translocations and point | Sporadic thyroid | Orphan receptor Tyr |
| | mutations | cancer; familial | Kinase |
| | | medullary thyroid | |
| | | cancer; multiple | |
| | | endocrine neoplasias | |
| | | 2A and 2B | |
| ROS | URII avian sarcoma | | Orphan receptor Tyr |
| | Virus | | Kinase |
| PDGF receptor | Translocation | Chronic | TEL(ETS-like |
| | | Myelomonocytic | transcription factor)/ |
| | | Leukemia | PDGF receptor gene |
| | | | Fusion |
| TGF-β receptor | | Colon carcinoma | |
| | | mismatch mutation | |
| | | target | |
| NONRECEPTOR | | | |
| TYROSINE KINASES | | | |
| ABI. | Abelson Mul.V | Chronic myelogenous | Interact with RB, |
| | | leukemia | RNA polymerase, |
| | | translocation | CRK, CBL |
| | | with BCR | |
| FPS/FES | Avian Fujinami SV; GA | | |
| | FeSV | | |
| LCK | Mul.V (murine leukemia | | Src family; T cell |
| | virus) promoter | | signaling; interacts |
| | insertion | | CD4/CD8 T cells |
| SRC | Avian Rous sarcoma | | Membrane-associated |
| | Virus | | Tyr kinase with |
| | | | signaling function; |
| | | | activated by receptor |
| | | | kinases |
| YES | Avian Y73 virus | | Src family; signaling |
| SER/THR PROTEIN | | | |
| KINASES | | | |
| AKT | AKT8 murine retrovirus | | Regulated by |
| | | | PI(3)K?; |
| | | | regulate 70-kd S6 k? |
| MOS | Maloney murine SV | | GVBD; cystostatic |
| | | | factor, MAP kinase |
| | | | kinase |
| PIM-1 | Promoter insertion | | |
| | Mouse | | |
| RAF/MIL | 3611 murine SV; MH2 | | Signaling in RAS |
| | avian SV | | Pathway |
| MISCELLANEOUS | | | |
| CELL SURFACE | | | |
| APC | Tumor suppressor | Colon cancer | Interacts with |
| | | | catenins |
| DCC | Tumor suppressor | Colon cancer | CAM domains |
| E-cadherin | Candidate tumor | Breast cancer | Extracellular |
| | Suppressor | | homotypic binding; |
| | | | intracellular |
| | | | interacts with |
| | | | catenins |
| PTC/NBCCS | Tumor suppressor and | Nevoid basal cell | 12 transmembrane |
| | Drosophilia homology | cancer syndrome | domain; signals |
| | | (Gorline | through Gli |
| | | syndrome) | homogue |
| | | | CI to antagonize |
| | | | hedgehog pathway |
| TAN-1 Notch | Translocation | T-ALI. | Signaling |
| homologue | | | |
| MISCELLANEOUS | | | |
| SIGNALING | | | |
| BCL-2 | Translocation | B-cell lymphoma | Apoptosis |
| CBL | Mu Cas NS-1 V | | Tyrosine- |
| | | | Phosphorylated |
| | | | RING |
| CRK | CT1010 ASV | | finger interact Abl |
| | | | Adapted SH2/SH3 |
| | | | interact Abl |
| DPC4 | Tumor suppressor | Pancreatic cancer | TGF-β-related |
| | | | signaling |
| | | | Pathway |
| MAS | Transfection and | | Possible angiotensin |
| | Tumorigenicity | | Receptor |
| NCK | | | Adaptor SH2/SH3 |
| GUANINE NUCLEOTIDE | | | |
| EXCHANGERS AND | | | |
| BINDING PROTEINS | | | |
| BCR | | Translocated | Exchanger; protein |
| | | with ABL | Kinase |
| | | in CML | |
| DBL | Transfection | | Exchanger |
| GSP | | | |
| NF-1 | Hereditary tumor | Tumor | RAS GAP |
| | Suppressor | suppressor | |
| | | neurofibromatosis | |
| OST | Transfection | | Exchanger |
| Harvey-Kirsten, | HaRat SV; Ki RaSV; | Point mutations | Signal cascade |
| N-RAS | Balb-MoMuSV; | in many | |
| | Transfection | human tumors | |
| VAV | Transfection | | S112/S113; |
| | | | exchanger |
| NUCLEAR PROTEINS | | | |
| AND TRANSCRIPTION | | | |
| FACTORS | | | |
| BRCA1 | Heritable suppressor | Mammary | Localization |
| | | cancer/ovarian | unsettled |
| | | cancer | |
| BRCA2 | Heritable suppressor | Mammary cancer | Function unknown |
| ERBA | Avian erythroblastosis | | Thyroid hormone |
| | Virus | | receptor |
| | | | (transcription) |
| ETS | Avian E26 virus | | DNA binding |
| EVII | MuLV promotor | AML | Transcription factor |
| | Insertion | | |
| FOS | FBI/FBR murine | | Transcription factor |
| | osteosarcoma viruses | | with c-JUN |
| GLI | Amplified glioma | Glioma | Zinc finger; cubitus |
| | | | interruptus homologue |
| | | | is in hedgehog |
| | | | signaling pathway; |
| | | | inhibitory link PTC |
| | | | and hedgehog |
| HMGI/LIM | Translocation t(3:12) | Lipoma | Gene fusions high |
| | t(12:15) | | mobility group |
| | | | HMGI-C (XT-hook) |
| | | | and transcription |
| | | | factor LIM or |
| | | | acidic domain |
| JUN | ASV-17 | | Transcription factor |
| | | | AP-1 with FOS |
| MLL/VHRX + | Translocation/fusion | Acute myeloid | Gene fusion of DNA- |
| ELI/MEN | ELL with MLL | leukemia | binding and methyl |
| | Trithorax-like gene | | transferase MLL |
| | | | with ELI RNA pol II |
| | | | elongation factor |
| MYB | Avian myeloblastosis | | DNA binding |
| | Virus | | |
| MYC | Avian MC29; | Burkitt's | DNA binding with |
| | Translocation B-cell | lymphoma | MAX partner; cyclin |
| | Lymphomas; promoter | | regulation; interact |
| | Insertion avian | | RB?; regulate |
| | leukosis | | apoptosis? |
| | Virus | | |
| N-MYC | Amplified | Neuroblastoma | |
| L-MYC | | Lung cancer | |
| REL | Avian | | NF-κB family |
| | Retriculoendotheliosis | | transcription factor |
| | Virus | | |
| SKI | Avian SKV770 | | Transcription factor |
| | Retrovirus | | |
| VHL | Heritable suppressor | Von Hippel- | Negative regulator or |
| | | Landau | elongin; transcriptional |
| | | syndrome | elongation complex |
| WT-1 | | Wilm's tumor | Transcription factor |
| CELL CYCLE/DNA | | | |
| DAMAGE RESPONSE | | | |
| ATM | Hereditary disorder | Ataxia- | Protein/lipid kinase |
| | | telangiectasia | homology; DNA |
| | | | damage response |
| | | | upstream in P53 |
| | | | pathway |
| BCL-2 | Translocation | Follicular | Apoptosis |
| | | lymphoma | |
| FACC | Point mutation | Fanconi's anemia | |
| | | group C | |
| | | (predisposition | |
| | | leukemia | |
| FHIT | Fragile site 3p14.2 | Lung carcinoma | Histidine triad-related |
| | | | diadenosine 5′,3″″- |
| | | | P¹•p⁴ tetraphosphate |
| | | | asymmetric hydrolase |
| hMLI/MutL | | HNPCC | Mismatch repair; |
| | | | MutL Homologue |
| HMSH2/MutS | | HNPCC | Mismatch repair, |
| | | | MutS Homologue |
| HPMS1 | | HNPCC | Mismatch repair; |
| | | | MutL Homologue |
| hPMS2 | | HNPCC | Mismatch repair, |
| | | | MutL Homologue |
| INK4/MTS1 | Adjacent INK-4B at | Candidate MTS1 | p16 CDK inhibitor |
| | 9p21; CDK complexes | suppressor and | |
| | | MLM melanoma gene | |
| INK4B/MTS2 | | Candidate | p15 CDK inhibitor |
| | | suppressor | |
| MDM-2 | Amplified | Sarcoma | Negative regulator |
| | | | p53 |
| p53 | Association with SV40 | Mutated >50% | Transcription factor; |
| | T antigen | human tumors, | checkpoint control; |
| | | including | apoptosis |
| | | hereditary Li- | |
| | | Fraumeni syndrome | |
| PRAD1/BCL1 | Translocation with | Parathyroid | Cyclin D |
| | Parathyroid hormone | adenoma; | |
| | or IgG | B-CLL | |
| RB | Hereditary | Retinoblastoma; | Interact cyclin/cdk; |
| | Retinoblastoma; | osteosarcoma; | regulate E2F |
| | Association with many | breast cancer; | transcription factor |
| | DNA virus tumor | other sporadic | |
| | Antigens | cancers | |
| XPA | | xeroderma | Excision repair, |
| | | pigmentosum; | photo- |
| | | skin cancer | product recognition; |
| | | predisposition | zinc finger |

### IV. Pharmaceutical Formulations, Delivery, and Treatment Regimens

In an embodiment of the present invention, compositions that induce stasis, are more effective under hypoxic conditions, or that have antitumor activity are contemplated.

An effective amount of the pharmaceutical composition, generally, is defined as that amount sufficient to detectably and repeatedly to ameliorate, reduce, minimize or limit the extent of the disease or its symptoms. More rigorous definitions may apply, including elimination, eradication or cure of disease.

Preferably, patients will have adequate bone marrow function (defined as a peripheral absolute granulocyte count of >2,000/mm³ and a platelet count of 100,000/mm³), adequate liver function (bilirubin<1.5 mg/dl) and adequate renal function (creatinine<1.5 mg/dl).

A. Administration

The routes of administration will vary, naturally, with the location and nature of the lesion, and include, e.g., intradermal, transdermal, parenteral, intravenous, intramuscular, intranasal, subcutaneous, percutaneous, intratracheal, intraperitoneal, intratumoral, perfusion, lavage, direct injection, and oral administration and formulation.

For anti-tumor therapy, intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. Multiple injections may be administered to the tumor, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising an antitumor compound. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be respectable. Treatments with therapeutic viral constructs may increase the respectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time.

Appropriate individual dosages for compositions of the present invention, including MITOTRACKER RED, include about 1 μg/kg, 25 μg/kg, 50 μg/kg, 75 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1.0 mg/kg, 1.25 mg/kg, 1.5 mg/kg, 1.75 mg/kg, 2.0 mg/kg, 2.25 mg/kg, 2.5 mg/kg and up to about 2.65 mg/kg.

B. Injectable Compositions and Formulations

The preferred method for the delivery of an antitumor compound of the present invention is via intratumoral injection. However, the pharmaceutical compositions disclosed herein may alternatively be administered parenterally, intravenously, intradermally, intramuscularly, transdermally or even intraperitoneally as described in U.S. Pat. No. 5,543,158; U.S. Pat. No. 5,641,515 and U.S. Pat. No. 5,399,363 (each specifically incorporated herein by reference in its entirety). Other compounds identified by screening methods of the invention may be employed as is described in any of the embodiments herein.

Injection of nucleic acid constructs may be delivered by syringe or any other method used for injection of a solution, as long as the expression construct can pass through the particular gauge of needle required for injection. A novel needleless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Induction of Stasis in Nematodes

Some metazoans have adapted to survive prolonged periods of oxygen deprivation (Storey, 1993). The brine shrimp Artemia Franciscana, marine mollusks, and Drosophila melanogaster are capable of surviving prolonged exposure to anoxia (0% O₂) (Storey, 1993; Foe et al., 1985). It has also been known that C. elegans in the dauer larvae stage of the life cycle are able to withstand the stress of anoxic exposure (Anderson, 1978).

A. Materials and Methods

2-cell C. elegans embryos were collected and exposed to either a normoxic environment (air, 20.8% O₂) or an environment that became anoxic (0% O₂) within 90 minutes (Time 0 is time when chamber became anoxic). The second time point is 24 hours after Time 0. Nematodes were visualized using differential interference contrast microscopy (also known as Nomarski optics). Images were collected and analyzed using NIH image and Adobe Photoshop 5.5. Embryos are approximately 50 μm in length.

L3 larvae were collected and exposed to either a normoxic or an anoxic environment for 24 hours. Nematodes were visualized using a dissecting microscope. Images were collected and analyzed using Metamorph and Adobe Photoshop 5.5.

Survival in anoxia for 24 hours, 48 hours, or 72 hours was determined for embryos, larvae, dauer larvae, and adult hermaphrodites at 20° C. Adult hermaphrodites were collected approximately 24 hours after L4 larvae stage. The data are representative of at least two independent experiments, and a total of greater than 400 nematodes each for post-embryonic stages and greater than 200 for embryos.

The presence of phosphoepitopes was determined in embryos exposed to normoxic or anoxic environment. Phosphoepitopes on some proteins are reduced in anoxia. Embryos were collected, exposed to a normoxic or anoxic environment, and stained with the DNA binding dye, 4′,6-diamidino-2-phenylindole (DAPI), a kinetochore protein (anti-HCP-1), mAb MPM-2, DAPI, phos SR, or phos H3 antibodies. Post-anoxic recovery was 1 hour. Western blot analysis of total proteins was done on embryos exposed to a normoxic or anoxic environment. Western blot was probed with phos H3 antibody and acetylated H3 antibody.

B. Results

All embryonic and post-embryonic nematodes exposed to an anoxic environment were found to enter a state of recoverable suspended animation. In the anoxic environment, embryo development stops. Upon re-exposure to a normoxic environment (20.8% O₂), embryos proceeded with development in a manner indistinguishable from normoxic embryos and developed into sexually mature adults. When post-embryonic nematodes were exposed to anoxia, they became immobile, stopped feeding, arrested larval development, and in the case of adults, did not lay eggs. Following the reintroduction of oxygen, nematodes resumed development within several hours.

The previous studies of Drosophila and Artemia have both shown that these organisms can only survive prolonged exposure to anoxia during specific time points in development (Storey, 1993; Foe et al., 1985). In contrast, C. elegans was found to survive anoxia exposure at all stages of their life cycle. Survival rate for a 24-hour exposure was 90% or greater. The capacity of nematodes to survive 48 hours or 72 hours of anoxia, however, decreased considerably—with the exception of L1 and dauer larvae, which maintained high viability.

C. elegans embryos exposed to anoxia were next compared with control embryos to characterize cell cycle changes associated with suspended animation. Embryos stained with the DNA-binding dye DAPI indicated that blastomeres of anoxic embryos arrested in interphase and at all stages of mitosis. This result was confirmed by staining embryos with an antibody specific for a kinetochore protein (HCP-1) that during mitosis exhibits dynamic changes in distribution during mitosis (Moore et al., 1999). These results contrast with studies of Drosophila, where embryos exposed to anoxia did not arrest at prometaphase or anaphase (Foe et al., 1985). In room air, DNA of C. elegans embryos is distributed throughout the nucleus in interphase. But after exposure to anoxia, the DNA was not uniformly distributed. This may indicate that entering suspended animation triggers a premature condensation of chromosomes, similar to what has been observed in Drosophila.

The ability of an organism to enter into and exit from suspended animation must involve a coordination of many cellular processes. Because protein phosphorylation is known to control basic cellular functions in a concerted fashion, whether the phosphorylation state of proteins is altered in nematodes exposed to anoxia was examined. Embryos were stained with antibodies that specifically bind the phosphorylated form of several different proteins including the SR protein splicing factors (Neugebauer et al., 1997), histone H3 (Hsu et al., 2000), and an antibody (MPM-2) that recognizes many phosphoproteins in mitotic cells (Davis et al., 1983). Phosphorylation of histone H3 at serine 10 increases during mitosis in a wide variety of organisms including C. elegans. After exposure of embryos to anoxia, antibody staining with MPM-2 and anti-phosphohistone H3 in mitotic cells was not detectable. In both cases the phosphoepitopes were detected when embryos were re-exposed to oxygen. In contrast staining was detected using anti-phospho SR antibody. These results suggest that regulation of the state of phosphorylation of certain proteins is important for entry into a state of recoverable suspended animation.

Several possibilities could account for the loss of staining, including loss of phosphate residues on the proteins, loss of the proteins, or sequestration of phosphoepitopes. To distinguish among these possibilities both the abundance and phosphorylation state of serine 10 on histone H3 was examined. The abundance of histone H3 was similar for embryos in normoxia and anoxia as seen by Coomassie staining and by Western blot analysis using an antibody that binds the acetylated form of histone H3. Additionally, the acetylated form of histone H3 was detected in embryos exposed to anoxia using indirect immunofluorescence. These results confirm that there is no loss of histone H3 in embryos arrested in anoxia. The phosphorylated form of histone H3, from embryos in anoxia was not detected. These results indicate that suspended animation is correlated with removal of phosphates on some proteins required for cell cycle and developmental progression.

### Example 2

### Induction of Stasis in Vertebrate Organism

A. Materials and Methods

Maintenance of Zebrafish. Zebrafish were raised as described (Popperl et al., 2000). Embryos were obtained by mating three females and two males. Embryos were carefully staged as described (Westerfield, 1995), and kept separate to create populations of synchronized embryos. For all experiments embryos were incubated in petri dishes with approximately 15 ml of fishwater at 28.5° C., unless otherwise stated.

Oxygen Deprivation Environments. For all studies an anaerobic bio-bag type A environmental chamber was used according to manufacturer's instructions (Becton Dickinson). This method contains a resazurin indicator that allows one to determine when the anoxic environment is established. A second method was used to verify suspended animation results. This method involved use of a chamber perfused with 100% N₂ gas (Airgas Inc.) and monitored for oxygen using a FYRITE O₂ Gas Analyzer (Bacharach, Pittsburgh, Pa.) and Resazurin Indicator (Becton Dickinson). By using these anoxia-producing methods, it took approximately two hours for the oxygen concentration to reach zero. Development of embryos could continue for another 1-2.5 hours, depending on the amount of water present. This suggests that a small amount of oxygen remained in the water during the first few hours of the experiment.

Viability Assays. Embryos were synchronized and collected during specific stages of embryogenesis and subjected to anoxia for 24 hours. Embryo viability was scored, upon return to a normoxic environment, by having the ability to develop to the larval stage with swim bladders. To control for the small number of embryos that die during embryogenesis, independent of oxygen availability, the viability of control embryos in normoxia were used as a standard to compare with embryos exposed to anoxia. At least 50 embryos, at the 2-cell stage or shield stage, which were subjected to 24 hours of anoxia, were allowed to recover in air and then raised to sexually mature adults. Fish from these populations were mated and determined to have the capacity to produce offspring.

Staining of Nuclei. 16-cell embryos were collected and placed into a petri dish with a small amount of fishwater, enough to keep embryos hydrated. This allowed a reduction in the time period until developmental arrest, which enabled better imaging of embryo nuclei. Control embryos (normoxia) were collected and fixed when the experimental embryos arrested development, approximately 2.5 hours after introduction into the anoxic producing chamber, at approximately the sphere stage of embryogenesis. Experimental embryos remained in the anoxic environment for 24 hours and were either immediately fixed (anoxia) or allowed to recover in air for 2 hours prior to fixation (post-anoxia). The method used to stain nuclei is similar to one previously described (Yager et al, 1997). Embryos were fixed in 4% formaldehyde in PBS for 3 hours, followed by a wash with, and incubation in, PBS. Embryos were dechorionated and deyolked carefully with forceps. The mass of embryonic cell caps were incubated with the DNA binding dye 4′,6-diamidino-2-phenylindole (DAPI) for approximately 20 minutes and washed once with Block buffer (3% BSA, 0.1% Tween, 2 mM MgCl₂ in PBS). Microscopy was done on a Zeiss Axioscope. Images were collected and analyzed using Adobe Photoshop 5.5. To estimate the number of blastomeres in interphase or mitosis, for anoxia exposed embryos in comparison to control embryos, random blastomeres from 4 embryos from each condition were counted, for a total of more than 1200 blastomeres.

Flow cytometric DNA content analysis. 4-cell embryos were collected and exposed to either a normoxic environment or an anoxic environment. Embryos exposed to anoxia arrested development at the shield stage of embryogenesis, approximately 4.5 hours after initiation of producing the anoxic environment. Embryos exposed to anoxia for 24 hours were either immediately analyzed (anoxia) or recovered in air for 2 hours before analysis (post-anoxia). Control embryos were analyzed at the shield stage of embryogenesis. The method used to analyze zebrafish DNA content by FACS was previously described (Zamir et al., 1997), with the exception that embryonic DNA was stained with DAPI. The nuclear suspensions were analyzed by the LSR flow cytometer (Becton Dickinson) and, the DNA histograms were analyzed by Cell Quest, and ModFit LT Ver. 2 (Verity Software House Inc.).

B. Results

Fully developed vertebrates that rely on circulatory systems instead of diffusion may be unable to adjust rapidly enough to survive anoxia. Here it is shown that zebrafish embryos exposed to an anoxic environment, in normal culture conditions and temperature, enter a state of suspended animation that can be maintained for 24 hours without deleterious effect. In the anoxic environment, development stopped. Upon re-exposure to a normoxic (20.8% O₂) environment, embryos continued with development. To determine whether exposure to anoxia caused any long term effects 100 embryos exposed to anoxia were raised to sexual maturity. These embryos were able to produce offspring and were indistinguishable from fish raised under normal conditions. This is the first time a vertebrate has been shown to arrest development in response to anoxia.

Several invertebrates that survive anoxia are only able to do so at specific times in development (Foe et al., 1985; Clegg, 1997). To define developmental stages when zebrafish embryos can survive anoxia, embryos at the periods of cleavage, blastula, gastrula, segmentation, straightening, and hatching were collected and subjected to anoxia for 24 hours (Kimmel et al., 1995). Zebrafish embryos 25 hours post-fertilization (h.p.f.) and younger were capable of surviving 24 hours of anoxia (Table 2). As embryos progress through development to the period of straightening (30 h.p.f.) the length of time that they could survive anoxia was reduced. Animals older than 48 h.p.f. were quite sensitive to anoxia (Table 2).

TABLE 2
Zebrafish Viability in Anoxia

| Period Stage | Percent Alive (N) |
|---|---|
| Cleavage Stage: | |
| 32/64 Cell | 83.1 (89) |
| Blastula: | |
| Oblong/Sphere | 83.2 (85) |
| Gastrula: | |
| Shield | 97.7 (90) |
| Segmentation: | |
| 10-Somite | 98.8 (85) |
| Straightening: | |
| 24 hpf~Prim-15 | 64 (100) |
| 29.5 hpf~Prim-15 | 4.4 (91) |

Zebrafish embryos exposed to anoxia had a great reduction in motility such as whole body movement and heartbeat For example, 29 h.p.f. embryos exposed to anoxia, displayed stopping of the heartbeat at 28.5° C., which normally beats at approximately 100 beats per minute (Baker et al. 1997). If these embryos were exposed to the anoxic environment for less than 8 hours, the heartbeat could return within several minutes upon exposure to oxygen. However, if the embryos were exposed to anoxia for 19 hours, it took approximately 6 hours of exposure to air for the heart rate to return to normal. Control-normoxic embryos exhibited heart rates similar to published data (Baker et al., 1997). There are rare situations in nature when heartbeat can cease for long periods of time without detrimental effects to the organism. For example, the freeze tolerant frogs (Rana sylvatica and Hyla veriscolor) and turtle (Chrysemys picta) display a stopping of heartbeat and blood flow at cold temperatures. Heartbeat is reestablished in these species upon thawing (Storey, 1990; Storey, 1997).

To determine if zebrafish embryos in a state of recoverable suspended animation arrest (stasis) at a specific point in the cell cycle we compared embryos exposed to anoxia with untreated embryos. Untreated embryos contained blastomeres with both mitotic and interphase nuclei (Yager et al. 1997). In contrast, blastomeres of anoxic embryos arrested in interphase but not mitosis. No mitotic cells were observed when embryos from other stages of development (25 and 30 h.p.f.) were placed into anoxia. In addition, the chromosomal DNA in anoxia treated embryos was found not to be uniformly distributed throughout the nucleus as it is in normal embryos. When arrested embryos were allowed to recover, they progressed in development with a frequency of mitotic cells comparable to untreated embryos. The fact that zebrafish embryos arrest in interphase and not mitosis contrasts with studies of Drosophila, where embryos exposed to anoxia arrest during interphase, prophase, metaphase, and telophase (Foe et al., 1985; DiGregorio et al., 2001).

To identify where in interphase arrest occurs DNA content was analyzed by flow cytometry analysis (FACS). The control embryos showed a characteristic cell cycle pattern for zebrafish embryos past the midblastula stage (Zamir et al., 1997). Surprisingly, the majority of cells from embryos exposed to anoxia arrested throughout S phase. G₀/G₁ cells appeared to be absent, and there were more blastomeres in the G₂ phase as compared to untreated embryos at the same stage of development. As expected, cells with G₁ DNA content were detected in the anoxia-exposed embryos after they were allowed to recover in normoxia for 2 hours. Together, the absence of mitosis observed by DAPI staining and the FACS analysis show that anoxia exposure causes cells to arrest in the S and G₂ phases of the cell cycle.

The amount of DNA in the arrested S phase nuclei was highly variable, indicating that there are many different points in S phase that arrest can occur. There are at least two possible explanations for this S and G₂ phase arrest. The first is that a checkpoint could be activated, in the S and G₂ phases, when oxygen levels are reduced. Alternatively, establishment of an anoxic environment takes about 4.5 hours and the average length of the cell cycle length is relatively short (approximately 80 minutes) at this time in development (Kane, 1999).

### Example 3

### Identification of Genes Required to Survive Anoxia

Programs of gene expression required for entry into or exit from anoxia-induced stasis that are conserved between vertebrates and invertebrates are identified. DNA microarrays of the complete C. elegans genome sequence (Washington University Genome Sequencing Center) and of the zebrafish expression sequence tag database arrays based on the zebrafish genome sequencing project (Beir, (1998) Genome Research 8:9-17) are used to examine gene expression at points throughout the stasis process. RNA is isolated from nematodes and fish at several time points during stasis, including point of entry (oxygen deprivation), stasis (anoxia), and exit (recovery). Positive hybridization of this RNA to the microarrays defines the genes expressed during this process.

### Example 4

### Functional Analysis of Identified Genes

Loss of function mutants are generated for each gene that exhibits increased expression during stasis or suspended animation using antisense approaches. Antisense morpholino oligonucleotides specific for each upregulated gene are injected into zebrafish eggs to phenocopy loss-of-function mutant alleles (Heasman et al., 2000).

RNA interference as generally described by Timmons et al. is used to generate loss of function mutants in C. elegans (Timmons et al., 1998). In this approach, double-stranded RNA (dsRNA) is expressed in bacteria and exposed to wild type animals by feeding them the bacteria to produce a loss-of-function allele of the gene in question (Zipperlen et al, 2000).

In C. elegans, the function of a candidate gene involved in the stasis or suspended animation pathway is tested by comparing the phenotypes found in room air with those found when the animals are challenged with anoxia and recovery. Double-stranded RNA bacteria made for a candidate gene is fed to nematodes for various lengths of time. The nematodes are observed for changes in viability, movement, fertility, and development. In parallel, dsRNA-treated worms are placed into an anoxic environment for 24 hours, released into room air, and scored for changes in viability, movement, fertility, and development.

Genes that appear to be required for suspended animation are further analyzed by direct visualization of tagged wild type worms mixed with dsRNA treated worms on a plate without food. The worms are observed to determine how the dsRNA treated worms move relative to the untreated worms as they enter into stasis. Double-stranded-treated worms that move when the control tagged wild-type worms have stopped moving suggests that the candidate gene is required for entry into stasis. Double-stranded RNA-treated worms that do not regain movement along with tagged wild-type controls when air is returned suggest that the candidate gene is required for exit from stasis.

The phosphorylation state of proteins are determined using the cytological tests described in Example 1.

Similarly, loss of function zebrafish embryos are analyzed using previously described assays to test for phenotypes related to exposure to anoxia to determine whether the candidate genes are required for entry or exit from stasis.

### Example 5

### Identification of Oxygen Sensor and Biomemetics

The induction of stasis upon hypoxic/anoxic conditions in the model systems described herein suggests the presence of an oxygen sensor that reports the concentration of available oxygen to the cell. When oxygen concentrations change, the sensor directs the cascade of events that lead to entry into or exit from stasis. The oxygen sensor is identified through the use of a genetic approach in C. elegans. In C. elegans a low threshold level of oxygen (0.5%) prevents nematodes from entering into suspended animation. Oxygen sensor mutants are identified by first mutagenizing nematodes and exposing the mutagenized animals to 0.5% oxygen for eighteen hours. During this time, all normal animals progress developmentally, whereas those with sensor mutations that cause them to precociously enter into suspended animation do not. Individuals containing such sensor mutations are identified, characterized and the oxygen sensor gene(s) is cloned and analyzed.

### Example 6

### Identification of Compounds Affecting Stasis

As described in more detail herein young zebrafish (25 hours or less post-fertilization) enter stasis when exposed to anoxia. During this time the survival rate can be as high as 98%. Shortly after this early period of development the rate of survival drops to approximately 4%, and by the time the embryos hatch (at 45 hours after fertilization) the fish have completely lost the ability to survive anoxia. Additionally, even when the young zebrafish are able to enter stasis, they are protected for only about 24 hours, after which viability begins to decrease with no survival after 72 hours.

a. Screen for Compounds that Permit Older Fish to Survive Anoxia

A compound library is screened to identify compounds that enable older fish to survive anoxia when they would otherwise die. Briefly, approximately 1 million compounds from a library of distinct, characterized organic compounds (Chembridge Corporation) in DMSO are evaluated at three different concentrations (1, 10, and 100 micromolar) to maximize the possibility of detecting dose-sensitive compounds.

For each screen, fish embryos at approximately 45 hours post fertilization (hpf) are treated with Pronase to remove the relatively impermeable chorion, the only barrier between the water and the cells. Between 3 to 5 pre-treated fish embryos are placed into each well of a 384-well plate. A different test compound is added to each well. The plates are incubated for one hour will to allow the drugs to take effect. The plates are exposed to anoxia as generally described in Example 2. Those test compounds that permit the 45 hpf embryos to survive anoxia are stasis inducer compounds.

b. Screen for Compounds that Prolong the Time that Fish Maintain Viability in Anoxia

A compound library is screened for compounds that prolong the time that young zebrafish can maintain viability in stasis. Compounds identified in each screen could have useful synergistic functions in inducing and maintaining stasis. Briefly, approximately 1 million compounds from a library of distinct, characterized organic compounds (Chembridge Corporation) in DMSO are evaluated at three different concentrations (1, 10, and 100 micromolar) to maximize the possibility of detecting dose-sensitive compounds.

For each screen, fish embryos at less than 25 hpf are treated with Pronase to remove the relatively impermeable chorion, the only barrier between the water and the cells. Between 3 to 5 pre-treated fish embryos are placed into each well of a 384-well plate. A different test compound is added to each well. The plates are incubated at 28° C. for at least one hour to permit the drugs to take effect. The plates are exposed to anoxia as generally described in Example 2 for three to four days at 28° C. Those test compounds that permit the 25 hpf embryos to survive anoxia after three to four days are stasis enhancing compounds.

An advantage of these screens is that compounds of interest will be identified by the presence of readily observable, living fish in those wells. In both screens, to score for viability after anoxia exposure, the compound are first be removed from the wells in an attempt to reverse stasis. The plates are incubated for several hours and examined for viability. A CCD camera system and a computer to screen are used to identify and record movement.

### Example 7

### Identification of Compounds Affecting Stasis in Mammals

This example is to identify chemicals that induce stasis in mammals. Such chemical compounds may be used to prevent injury resulting from oxygen deprivation, which occurs during trauma and some surgical procedures. Mouse blastocysts can enter stasis under certain natural conditions (known as diapause). Mouse embryos do not normally enter stasis in vivo. Mouse blastocysts are isolated at a stage when they can enter stasis. Compounds are added to the embryos to determine whether the compounds induce stasis in the embryos Direct assays of developmental and cell cycle progression are used to examine whether stasis was induced.

Induction of stasis may involve many changes in gene expression. To avoid missing possible synergistic interactions between compounds that may be required to activate stasis, patterns of gene expression from mouse embryos in natural stasis (diapause) are compared with patterns obtained from embryos exposed to test compounds.

It is possible that positive compounds found in zebrafish will need to be optimized to allow full benefit to be realized in mammals. Chemical derivatives may be synthesized. This process of lead compound optimization will involve combinatorial organic chemistry to generate libraries of compounds related to the initially positive compounds. Such libraries will then be used in the mouse to optimize for the desired effect.

### Example 8

### Mitotracker Red

This example describes the identification of a compound that causes fish embryos subjected to an anoxic/hypoxic environment to die. In this example, fish were maintained at 28° C. in fish water or embryo media (Westerfield, 1995). Briefly, zebrafish embryos at approximately 8 hpf were pre-treated with Pronase to remove the chorion. Approximately 10 pre-treated fish were placed in each 30 mm petri dishes in either fish water or embryo media. Dilutions of MITOTRACKER RED (Molecular Probes, Eugene Oreg.) were added to the pre-treated embryos in concentrations from approximately 10 μg/ml reduced in half-logs concentrations down to approximately 1 ng/ml. The embryos were exposed to the compound for approximately two hours at 28° C. After incubation with the compound, the petri-dishes were placed in BIO-BAGS (Becton Dickinson) to produce anoxic conditions in the dishes. The embryos were incubated for 24 hours at 28° C. As a control, pre-treated embryos treated with MITOTRACKER RED were incubated in normoxic conditions at 28° C. for 24 hours. After 24 hours in anoxia, the embryos were returned to room air and viability was assessed visually. The MITOTRACKER RED-treated embryos at a concentration of 500 nM were not viable after anoxia while those embryos treated with MITOTRACKER RED, but not placed in anoxia were viable.

### Example 9

### Use of Mitotracker Red

This example describes the treatment of mice bearing human tumor nodules. Eight NOD SCID mice are inoculated with 3×10⁷ 8226 human myeloma cells by interscapular subcutaneous injection. Palpable tumor nodules are measured in three dimensions with calipers. Serum from injected mice are tested for the presence of Human lambda light chain indicating the presence of the human myeloma cells. Four mice are treated with 265 micrograms per kilogram of MITOTRACKER RED in 100 microliters by tail vein injection on days 8, 11 and 14. Control mice receive injections of saline without MITOTRACKER RED. Tumor nodule size in three dimensions is measured in all mice for 14 days. Mice are re-dosed with 265 micrograms per kilogram MITOTRACKER RED on days 26, 28, 29 and 33. Decrease in nodule size is indicative of regression of the tumor.

### Example 10

### Identification of New Stasis Inducers

This example describes the identification of a compound that causes fish embryos to enter stasis. In this example, fish were maintained at 28° C. in fish water or embryo media. Briefly, zebrafish embryos at approximately 8 hpf were pre-treated with Pronase to remove the chorion. Approximately 10 pre-treated fish were placed in 30 mm petri dishes in either fish water or embryo media. Dilutions of Rotenone (Sigma, St. Louis, Mo.) were added to the pre-treated fish in concentrations from approximately 10 μg/ml reduced in half-logs concentrations down to approximately 10 ng/ml. The embryos were exposed to the compound for approximately 12 hours at 28° C. under normoxic conditions. As a control, pre-treated embryos were incubated in normoxic conditions at 28° C. for the same period of time. After incubation with the compound, the embryos were assessed visually for entry into stasis. The embryos treated with 0.1 μg/ml of Rotenone were shown to enter stasis as evidenced by characteristics described in Example 2. The Rotenone-treated embryos were removed into fresh medium and incubated at 28° C. under normoxic conditions for 24 hours. The embryos were then assessed visually for the ability to exit stasis. It was found that Rotenone is a reversible stasis inducer of zebrafish embryos.

### Example 11

### Treatment with Rotenone

This example describes the treatment of mouse blastocysts with rotenone to mimic mouse diapause. 4.5 day-old mouse blastocysts are harvested and placed in blastocyst culture medium M26 (SIGMA) or in blastocyst culture medium containing Rotenone in concentrations from approximately 10 μg/ml reduced in half-logs concentrations down to approximately 10 ng/ml. The blastocysts are incubated at 37° C. for 24 hours. After incubations, the blastocysts are examined for evidence of diapause by the absence of development and cell division. Rotenone-treated blastocysts are removed to fresh medium to permit the blastocysts to exit stasis and subsequently implanted into pseudo-pregnant mice to determine whether the arrested blastocysts are viable.

All of the compositions and methods disclosed and claimed herein can be made and executed without undue experimentation in light of the present disclosure. While the compositions and methods of this invention have been described in terms of embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
EPA No. 320 308
EPA No. 329 822
GB Application No. 2,202,328
GB Application No. 2193095
PCT/US85/01161
PCT/US87/00880
PCT/US89/01025
PCT/US89/05040
U.S. Pat. No. 3,817,837
U.S. Pat. No. 3,850,752
U.S. Pat. No. 3,939,350
U.S. Pat. No. 3,996,345
U.S. Pat. No. 4,162,282
U.S. Pat. No. 4,196,265
U.S. Pat. No. 4,275,149
U.S. Pat. No. 4,277,437
U.S. Pat. No. 4,310,505
U.S. Pat. No. 4,366,241
U.S. Pat. No. 4,533,254
U.S. Pat. No. 4,554,101
U.S. Pat. No. 4,559,258
U.S. Pat. No. 4,683,195
U.S. Pat. No. 4,683,202
U.S. Pat. No. 4,684,611
U.S. Pat. No. 4,728,575
U.S. Pat. No. 4,728,578
U.S. Pat. No. 4,737,323
U.S. Pat. No. 4,800,159
U.S. Pat. No. 4,879,236
U.S. Pat. No. 4,883,750
U.S. Pat. No. 4,921,706
U.S. Pat. No. 4,946,773
U.S. Pat. No. 4,952,500
U.S. Pat. No. 5,054,297
U.S. Pat. No. 5,217,860
U.S. Pat. No. 5,279,721
U.S. Pat. No. 5,288,514
U.S. Pat. No. 5,302,523
U.S. Pat. No. 5,322,783
U.S. Pat. No. 5,354,855
U.S. Pat. No. 5,384,253
U.S. Pat. No. 5,399,363
U.S. Pat. No. 5,464,765
U.S. Pat. No. 5,466,468
U.S. Pat. No. 5,538,877
U.S. Pat. No. 5,538,880
U.S. Pat. No. 5,543,158
U.S. Pat. No. 5,550,318
U.S. Pat. No. 5,563,055
U.S. Pat. No. 5,580,859
U.S. Pat. No. 5,589,466
U.S. Pat. No. 5,591,616
U.S. Pat. No. 5,609,870
U.S. Pat. No. 5,610,042
U.S. Pat. No. 5,641,515
U.S. Pat. No. 5,656,610
U.S. Pat. No. 5,693,762
U.S. Pat. No. 5,702,932
U.S. Pat. No. 5,736,524
U.S. Pat. No. 5,739,169
U.S. Pat. No. 5,780,448
U.S. Pat. No. 5,785,970
U.S. Pat. No. 5,789,215
U.S. Pat. No. 5,801,005
U.S. Pat. No. 5,824,311
U.S. Pat. No. 5,830,880
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,225
U.S. Pat. No. 5,846,233
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,849,481
U.S. Pat. No. 5,849,483
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,770
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,155
U.S. Pat. No. 5,861,244
U.S. Pat. No. 5,863,732
U.S. Pat. No. 5,863,753
U.S. Pat. No. 5,866,331
U.S. Pat. No. 5,866,337
U.S. Pat. No. 5,866,366
U.S. Pat. No. 5,871,986
U.S. Pat. No. 5,879,703
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,925,525
U.S. Pat. No. 5,925,565
U.S. Pat. No. 5,928,862
U.S. Pat. No. 5,928,869
U.S. Pat. No. 5,928,870
U.S. Pat. No. 5,928,905
U.S. Pat. No. 5,928,906
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,819
U.S. Pat. No. 5,935,825
U.S. Pat. No. 5,939,291
U.S. Pat. No. 5,942,391
U.S. Pat. No. 5,945,100
U.S. Pat. No. 5,952,168
U.S. Pat. No. 5,980,912
U.S. Pat. No. 5,981,274
U.S. Pat. No. 5,994,624
U.S. Pat. No. 6,020,192
U.S. Pat. No. 6,027,727
U.S. Pat. No. 6,153,375
U.S. Pat. No. 6,168,912
U.S. Pat. No. 6,184,389
U.S. Pat. No. 6,185,506
U.S. Pat. No. 6,187,529
U.S. Pat. No. 6,194,137
WO 84/03564
WO 88/10315
WO 89/06700
WO 90/07641
WO 93/09668 (PCT/US92/10183)
WO 94/09699
WO 95/06128
WO 99/18933
Abbondanzo et al., Breast Cancer Res. Treat., 16:182(#151), 1990.
Allred et al., Breast Cancer Res. Treat., 16:182(#149), 1990.
Almendro et al., J Immunol., 157:5411-5421, 1996.
Altschul et al, Nucleic Acids Res., 25:3389-3402, 1997.
Altschul, S. F., & Koonin, E. V. (1998) Trends Biochem. Sci. 11, 444-447.
Anderson, Can. J. Zool., 56:1786-1791 (1978).
Angel et al., Cell, 49:729, 1987b.
Angel et al., Mol. Cell. Biol., 7:2256, 1987a.
Araki et al., Proc. Natl. Acad. Sci. USA 92: 160-164, 1995.
Arap et al., Cancer Res., 55:1351-1354, 1995.
Aravind, L., Walker, D. R., & Koonin, E. V. (1999) Nucleic Acids Res. 27, 1223-1242.
Atchison and Perry, Cell, 46:253, 1986.
Atchison and Perry, Cell, 48:121, 1987.
Atherton, et al., Solid Phase Peptide Synthesis: A Practical Approach (IRL Press: Oxford, 1989).
Austin-Ward, Villaseca, Rev. Med. Chil., 126(7):838-45, 1998.
Ausubel, ed., Current protocols in molecular biology, New York, John Wiley & Sons, 1996.
Baichwal and Sugden, In: Gene Transfer, Kucherlapati R, ed., New York, Plenum Press, pp. 117-148, 1986.
Bailly, V., Lauder, S., Prakash, S., & Prakash, L. (1997) J. Biol. Chem. 272, 23360-23365.
Bajorin et al., Proc. Annu. Meet. Am. Soc. Clin. Oncol., 7:A967, 1988.
Baker, G. et al. (eds.), Modern Pharmaceutics, Marcel Dekker, Inc., New York, N.Y., 1990.
Baker, et al., Proc. Natl. Acad. Sci. U.S.A. 94:4554-9, 1997).
Bakhshi et al., Cell, 41(3):899-906, 1985.
Baldwin, et al., J. Am. Chem. Soc. 117:5588 (1995).
Banerji et al., Cell, 27:299, 1981.
Banerji et al., Cell, 35:729, 1983.
Bangham, et al., J. Mol. Biol., 13:238-252, 1965.
Bebenek et al., J. Biol. Chem., 270:19516-19523, 1995.
Bebenek et al., Methods Enzymol, 262:217-232, 1995.
Beebe, et al., J. Am. Chem. Soc. 114:10061 (1992).
Beir, Genome Research 8:9-17 (1998).
Bell et al., J. Biol. Chem., 272:7345-7351, 1997.
Benjamini, “Immunology: A Short Course,” Wiley-Liss, New York (3rd ed., 1991).
Berkhout et al., Cell, 59:273, 1989.
Berzal-Herranz, A. et al., Genes and Devel., 6:129-134, 1992.
Blanar et al, EMBO J, 8:1139, 1989.
Bock, et al, Nature 355:564 (1992).
Bodine and Ley, EMBO J, 6:2997, 1987.
Bonavida et al., Int J Oncol, 15:793-802, 1999.
Bonavida et al., Proc Nat'l Acad Sci USA. 97:1754-9, 2000.
Borden K. L., & Freemont, P. S. (1996) Curr. Opin. Struct. Biol. 6, 395-401.
Boshart et al., Cell, 41:521, 1985.
Bosze et al., EMBO J, 5:1615, 1986.
Braddock et al., Cell, 58:269, 1989.
Braithwaite, D. K., & Ito, J. (1993) Nucleic Acids Res 21, 787-802.
Brenner, et al., Proc. Natl. Acad. Sci. USA. 89:5381 (1992).
Brotcorne-Lannoye and Maenhaut-Michel, Proc. Natl. Acad. Sci. USA 83: 3904-3908, 1986.
Brown, Cancer Res. 59:5863 (1999).
Brown et al. Breast Cancer Res. Treat., 16:192(#191), 1990.
Brutlag et al., CABIOS, 6:237-245, 1990.
Bukowski et al., Clin. Cancer Res., 4(10):2337-47, 1998.
Bulla and Siddiqui, J. Virol., 62:1437, 1986.
Bunin, et al., J. Am. Chem. Soc. 114:10997 (1992).
Bunin, et al., Proc. Natl. Acad. Sci. USA. 91:4708 (1994).
Burg, et al., Antimicrob. Agents Chemother, 15:361 (1979).
Caldas et al., Nat. Genet., 8:27-32, 1994.
Campbell, et al., Mol. Cell. Biol., 8:1993, 1988.
Campbell, et al., J. Am. Chem. Soc. 117:5381 (1995).
Campbell, In: Monoclonal Antibody Technology, Laboratory Techniques in Biochemistry and Molecular Biology, Vol. 13, Burden and Von Knippenberg, Eds. pp. 75-83, Amsterdam, Elseview, 1984.
Campere and Tilghman, Genes and Dev., 3:537, 1989.
Campo et al., Nature, 303:77, 1983.
Canfield et al., Methods in Enzymology, 189, 418-422, 1990.
Capaldi et al., Biochem. Biophys. Res. Comm., 76:425, 1977.
Carbonelli et al. FEMS Microbiol Lett. 177(1):75-82, 1999.
Cech et al., Cell, 27:487-496, 1981.
Celander and Haseltine, J Virology, 61:269, 1987.
Celander et al., J Virology, 62:1314, 1988.
Chandler et al., Cell, 33:489, 1983.
Chandler et al., Proc Natl Acad Sci USA. 94(8):3596-3601, 1997.
Chang et al., Mol. Cell. Biol., 9:2153, 1989.
Chatterjee et al., Proc. Nat. Acad. Sci. USA., 86:9114, 1989.
Chen, et al., Mol. Cell. Biol., 7:2745-2752, 1987.
Chen, et al., J. Am. Chem. Soc. 116:2661 (1994).
Cheng et al., Cancer Res., 54:5547-5551, 1994.
Cheng, et al., Investigative Radiology, vol. 22, pp. 47-55 (1987).
Cho, et al., Science 261:1303 (1993).
Choi et al., Cell, 53:519, 1988.
Chou and Fasman, Adv. Enzymol. Relat. Areas Mol. Biol., 47:45-148, 1978a.
Chou and Fasman, Ann. Rev. Biochem., 47:251-276, 1978b.
Chou and Fasman, Biochemistry, 13(2):222-245, 1974a.
Chou and Fasman, Biophys. J, 26:367-384, 1979.
Chowrira, B. H. et al., Biochemistry, 32:1088-1095, 1993.
Chowrira, B. H. et al., J. Biol. Chem., 269:25856-25864, 1994.
Christodoulides et al., Microbiology, 144(Pt 11):3027-37, 1998.
Clark et al., J. Mol. Biol. 198, 123-127, 1987.
Cleary and Sklar, Proc. Nat'l. Acad. Sci. USA, 82(21):7439-43, 1985.
Cleary et al., J. Exp. Med., 164(1):315-20, 1986.
Clegg, J. Exp. Biol., 200:467 (1997).
Clementi, et al., Proc. Natl. Acad. Sci. USA. 96:1559 (1999).
Cocea, Biotechniques. 23(5):814-816, 1997.
Cohen et al., J. Cell. Physiol., 5:75, 1987.
Costa et al., Mol. Cell. Biol., 8:81, 1988.
Coupar et al., Gene, 68:1-10, 1988.
Cripe et al., EMBO J., 6:3745, 1987.
Culotta and Hamer, Mol. Cell. Biol., 9:1376, 1989.
Culver et al., Science, 256:1550-1552, 1992.
Cummings and Zoghbi, Hum. Mol. Genet., 9:909-16, 2000.
Cwirla, et al., Proc. Natl. Acad. Sci. U.S.A. 87:6378 (1990).
Dandolo et al., J. Virology, 47:55, 1983.
Dankwardt, et al., Tetrahedron Lett. 36:4923 (1995).
Davidson et al., J. Immunother., 21(5):389-98, 1998.
Davis, et al., Proc. Natl. Acad. Sci. U.S.A. 80:2926 (1983).
De Villiers et al., Nature, 312:242, 1984.
Deamer and P. Uster, Liposomes (M. Ostro, ed.), Marcel Dekker, Inc., New York, pp. 27-52, 1983.
Dejager et al., J. Clin. Invest., 92:894-902, 1993.
Deprez, et al, J. Am. Chem. Soc. 117:5405 (1995).
Deschamps et al., Science, 230:1174, 1985.
Devlin, et al., Science, 249:404 (1990).
DeWitt, et al., Proc. Natl. Acad. Sci. U.S.A. 90:6909 (1993).
Diaz et al., Int. Immun. 11: 825-833, 1999.
Dillman Cancer Biother. Radiopharm., 14:5-10, 1999.
Doolittle et al., Methods Mol. Biol., 109:215-37, 1999.
Dudley, et al., Proc. Natl. Acad. Sci. USA., 92:7686 (1995).
Edbrooke et al., Mol. Cell. Biol., 9:1908, 1989.
Edlund et al., Science, 230:912, 1985.
Eichler, et al., Peptide Res. 4:296 (1991).
El-Gorab et al., Biochem. Biophys. Acta, 1973, 306, 58-66, 1973.
Englebretsen, et al, Int. J. Peptide Protein Res. 40:487 (1992).
Erb, et al., Proc. Natl. Acad. Sci. USA. 91:11422 (1994).
Esposito et al., Proc. Natl. Acad. Sci. USA 97: 1166-1171, 2000.
Feaver et al., J. Biol. Chem., 2000.
Fechheimer et al., Proc. Nat'l Acad Sci. USA, 84:8463-8467, 1987.
Felsenstein, Methods Enzymol. 266, 418-427, 1996.
Fendler et al., Catalysis in Micellar and Macromolecular Systems, Academic Press, New York, 1975.
Feng and Holland, Nature, 334:6178, 1988.
Fetrow and Bryant, Biotech., 11:479-483, 1993.
Firak and Subramanian, Mol. Cell. Biol., 6:3667, 1986.
Fodor et al., Science, 251:767-773, 1991.
Foe, et al., J. Cell Biol. 100:1623-1636 (1985).
Foecking and Hofstetter, Gene, 45(1):101-5, 1986.
Forman, et al, J. Org. Chem. 60:523 (1995).
Forster and Symons, Cell, 49:211-220, 1987.
Fraley et al., Proc. Nat'l Acad. Sci. USA, 76:3348-3352, 1979.
Frank, Bioorg. Med. Chem. Lett. 3:425 (1993).
Frank, et al., Tetrahedron 44:031 (1988).
Friedberg & Gerlach, Cell, 98:413-6, 1999.
Friedberg & Gerlach, Cell, in press, 1999.
Friedberg et al., DNA Repair and Mutagenesis (Am. Soc. Microbiol., Washington, D.C.), 1995.
Friedmann, Science, 244:1275-1281, 1989.
Frohman, In: PCR Protocols: A Guide To Methods And Applications, Academic Press, N.Y., 1990.
Fujita et al., Cell, 49:357, 1987.
Furka, et al., Abst. 14^{th} Intl. Congress Biochem., Prague 5:47 (1988).
Furka, et al., Int. J. Peptide Protein Res. 37:487 (1991).
Gabizon et al., Cancer Res., 50(19):6371-8, 1990.
Gallop, M. A., et al, J Med. Chem. 37:1385 (1994)
Gerlach et al., Proc. Natl. Acad. Sci. USA 96: 11922-11927, 1999.
Gerlach et al., Nature (London), 328:802-805, 1987.
Geysen, H. M., et al., Proc. Natl. Acad. Sci. U.S.A. 81:3998 (1984).
Ghose and Blair, Crit. Rev. Ther. Drug Carrier Syst., 3(4):263-359, 1987.
Ghosh and Bachhawat, In: Liver diseases, targeted diagnosis and therapy using specific receptors and ligands, (Wu G, Wu C ed.), New York: Marcel Dekker, pp. 87-104, 1991.
Gilles et al., Cell, 33:717, 1983.
Giulivi, Biochem. J, 332:673 (1998).
Gliniak et al., Cancer Res. 59:6153-8, 1999.
Gloss et al, EMBO J., 6:3735, 1987.
Godbout et al., Mol. Cell. Biol., 8:1169, 1988.
Goodbourn and Maniatis, Proc. Nat'l Acad Sci. USA, 85:1447, 1988.
Goodbourn et al., Cell, 45:601, 1986.
Gopal, Mol. Cell Biol., 5:1188-1190, 1985.
Gordon, et al., J. Bioorg. Med. Chem. Lett. 5:47 (1995).
Graham and Van Der Eb, Virology, 52:456-467, 1973.
Greene et al., Immunology Today, 10:272, 1989.
Gregoriadis, ed., Drug Carriers In Biology And Medicine, pp. 287-341, 1979.
Gregoriadis, G., ed., Liposome Technology, vol. 1, pp. 30-35, 51-65 and 79-107 (CRC Press Inc., Boca Raton, Fla., 1984.
Grosschedl and Baltimore, Cell, 41:885, 1985.
Grubler, et al., in Peptides: Chemistry, Structure, and Biology (Proceedings of the Thirteenth American Peptide Symposium) (Hodges and Smith, Eds., ESCOM-Leiden, The Netherlands, 1994).
Gu et al., Science 265: 103-106, 1994.
Guillemin, et al., Cell, 89, 9-12, 1997.
Gulbis et al., Hum. Pathol., 24:1271-85, 1993.
Hacia et al., Nature Genetics, 14:441-447, 1996.
Hand, in: Surviving hypoxia mechanisms of control and adaptation, ed. Hochachka, et al (CRC Press, Inc., Boca Raton, pp. 171-185 (1993).
Hanibuchi et al., Int. J. Cancer, 78(4):480-5, 1998.
Harland and Weintraub, J. Cell Biol., 101:1094-1099, 1985.
Harlow and Lane, “Antibodies: A Laboratory Manual,” Cold Spring Harbor Laboratory, 1988.
Haseloff and Gerlach, Nature, 334:585-591, 1988.
Haslinger and Karin, Proc. Nat'l Acad Sci. USA., 82:8572, 1985.
Hauber and Cullen, J. Virology, 62:673, 1988.
Heasman, et al., Dev. Biol. 222(1): 124 (2000).
Hellstrand et al., Acta. Oncol., 37(4):347-53, 1998.
Hen et al., Nature, 321:249, 1986.
Hensel et al., Lymphokine Res., 8:347, 1989.
Hermonat and Muzyczka, Proc. Nat'l. Acad. Sci. USA, 81:6466-6470, 1984.
Herr and Clarke, Cell, 45:461, 1986.
Hindges, and Hübscher, Biol. Chem. 378, 345-362, 1997.
Hirochika et al., J. Virol., 61:2599, 1987.
Hirsch et al., Mol. Cell. Biol., 10: 1959, 1990.
Hochachka, Science, 231:234 (1986)
Hochachka, et al., Proc. Natl. Acad. Sci. U.S.A. 93:9493 (1996).
Hochachka, et al., Surviving hypoxia mechanisms of control and adaptation (CRC Press, Inc., Boca Raton) (1993).
Holbrook et al., Virology, 157:211, 1987.
Hollstein et al., Science 253:49-53, 1991.
Hope et al., Biochimica et Biophysica Acta, 812: 55-65, 1985.
Horlick and Benfield, Mol. Cell. Biol., 9:2396, 1989.
Horwich et al. J. Virol., 64:642-650, 1990.
Houghten, et al, Nature 354:84 (1991).
Houghten, et al., Proc. Natl. Acad. Sci. USA. 82:5131 (1985).
Hsu, et al, Cell 102:279 (2000).
Huang et al., Cell, 27:245, 1981.
Hug et al, Mol Cell Biol., 8:3065, 1988.
Hui and Hashimoto, Infect. Immun., 66(11):5329-36, 1998.
Hussussian et al., Nature Genetics, 15-21, 1994.
Hwang et al., Mol. Cell. Biol., 10: 585, 1990.
Imagawa et al., Cell, 51:251, 1987.
Imbra and Karin, Nature, 323:555, 1986.
Imler et al, Mol. Cell. Biol., 7:2558, 1987.
Imperiale and Nevins, Mol. Cell. Biol., 4:875, 1984.
Inouye et al., Nucleic Acids Res., 13:3101-3109, 1985.
Irie & Morton, Proc. Nat'l Acad Sci. USA 83:8694-8698, 1986 Irie et al., “Melanoma gangliosides and human monoclonal antibody,” In: Human Tumor Antigens and Specific Tumor Therapy, Metzgar & Mitchell (eds.), Alan R. Liss, Inc., New York, pp. 115-126, 1989.
Iyer, et al., Genes Dev., 12:149 (1998).
Jacobs et al., J. Exp. Med. 187: 1735-1743, 1998.
Jakobovits et al., Mol. Cell. Biol., 8:2555, 1988.
Jameel and Siddiqui, Mol. Cell. Biol., 6:710, 1986.
Jameson and Wolf, Comput. Appl. Biosci., 4(1):181-186, 1988.
Janda, K. D., Proc. Natl. Acad. Sci. U.S.A. 91:10779 (1994).
Jaynes et al, Mol. Cell. Biol., 8:62, 1988.
Johnson et al., J. Biol. Chem. 274: 15975-15977, 1999c.
Johnson et al., J. Biol. Chem. 275:7447-7450, 2000b.
Johnson et al., J. Virol., 67:438-445, 1993.
Johnson et al., Mol. Cell. Biol., 9:3393, 1989.
Johnson et al., Proc. Natl. Acad. Sci. USA 97: 3838-3843, 2000.
Johnson et al, Science 283, 1001-1004, 1999a.
Johnson et al., Science 285, 263-265, 1999b.
Jones et al., Nucleic Acids Res. 25, 7119-7131, 1988.
Joyce, Nature, 338:217-244, 1989.
Ju et al., Gene Ther., 7(4):329-38, 2000.
Kadesch and Berg, Mol. Cell. Biol., 6:2593, 1986.
Kaeppler et al., Plant Cell Reports 9: 415-418, 1990.
Kamb et al., Nature Genetics, 8:22-26, 1994.
Kamb et al., Science, 2674:436-440, 1994.
Kaneda et al., Science, 243:375-378, 1989.
Karin et al., Mol. Cell. Biol., 7:606, 1987.
Katinka et al., Cell, 20:393, 1980.
Katinka et al., Nature, 290:720, 1981.
Kato et al, J. Biol. Chem., 266:3361-3364, 1991.
Kawamoto et al, Mol. Cell. Biol., 8:267, 1988.
Kayser, et al., Anesthesiology 90:545 (1999).
Keane et al., Cancer Res. 59:734-41, 1999.
Kerr et al., Br. J. Cancer, 26(4):239-57, 1972.
Kerr, et al., J. Am. Chem. Soc. 115:2529 (1993).
Kick, et al., J Med. Chem. 38:1427 (1995).
Kiledjian et al., Mol. Cell. Biol., 8:145, 1988.
Kim and Cech, Proc. Nat'l Acad Sci. USA, 84:8788-8792, 1987.
Kim et al., Proc. Natl. Acad. Sci. USA 94: 13792-13797, 1997.
Kimmel, et al., Dev. Dyn. 203:253-310, 1995).
Kiamut et al., Mol. Cell. Biol., 10: 193, 1990.
Koch et al., Mol. Cell. Biol., 9:303, 1989.
Kozak, J. Cell Biol. 108, 229-241, 1989.
Kraemer et al., Mutat. Res. 220: 61-72, 1989.
Kraus et al. FEBS Lett., 428(3):165-170, 1998.
Kriegler and Botchan, In: Eukaryotic Viral Vectors, Y. Gluzman, ed., Cold Spring Harbor: Cold Spring Harbor Laboratory, NY, 1982.
Kriegler and Botchan, Mol. Cell. Biol., 3:325, 1983.
Kriegler et al., Cell, 38:483, 1984a.
Kriegler et al., Cell, 53:45, 1988.
Kriegler et al., In: Cancer Cells 2/Oncogenes and Viral Genes, Van de Woude et al. eds, Cold Spring Harbor, Cold Spring Harbor. Laboratory, 1984b.
Kriegler et al., In: Gene Expression, D. Hamer and M. Rosenberg, eds., New York: Alan R. Liss, 1983.
Kuhl et al., Cell, 50:1057, 1987.
Kulaeva et al., Mut. Res. 357, 245-253, 1996.
Kunkel & Bebenek, Annu. Rev. Biochem., 69, in press, 2000.
Kunkel, J. Biol. Chem., 261:13581-13587, 1986.
Kunz et al., Nucl. Acids Res., 17:1121, 1989.
Kurth, et al., J. Org. Chem. 59:5862 (1994).
Kwoh et al., Proc. Nat. Acad. Sci. USA, 86: 1173, 1989.
Kyte and Doolittle, J. Mol. Biol., 157(1):105-132, 1982.
Lam, K. S., et al., Nature, 354:82 (1991).
Landis et al., CA Cancer J. Clin., 48:6, 1998.
Lareyre et al., J Biol Chem., 274(12):8282-8290, 1999.
Latimer et al., J. Bacteriol. 171, 230-237, 1989.
Larsen et al., Proc. Nat'l Acad. Sci. USA., 83:8283, 1986.
Laspia et al., Cell, 59:283, 1989.
Latimer et al., Mol. Cell. Biol., 10:760, 1990.
Lee et al., J Auton Nerv Syst. 74(2-3):86-90, 1997.
Lee et al., Nature, 294:228, 1981.
Lee et al., Nucleic Acids Res., 12:4191-206, 1984.
Levenson et al., Hum Gene Ther. 20; 9(8):1233-1236, 1998.
Levine, The Molecular Basis of Cancer, Mendelsohn, et al., eds. WB Saunders Co., Philadelphia, 1995.
Levinson et al., Nature, 295:79, 1982.
Li & Herskowitz, Science 262, 1870-1874, 1993
Lieber and Strauss, Mol. Cell. Biol., 15: 540-551, 1995.
Lin et al., Mol. Cell. Biol., 10:850, 1990.
Luria et al, EMBO J., 6:3307, 1987.
Lusky and Botchan, Proc. Nat'l Acad. Sci. USA., 83:3609, 1986.
Lusky et al., Mol. Cell. Biol., 3:1108, 1983.
Macejak and Sarnow, Nature, 353:90-94, 1991.
Majors and Varmus, Proc. Nat'l Acad. Sci. USA., 80:5866, 1983.
Marsters et al., Recent Prog Horm Res 54:225-34, 1999.
Martin et al., Nature, 345(6277):739-743, 1990.
Masutani et al., EMBO J. 18, 3491-3501, 1999.
Masutani et al., EMBO J. 18: 3491-3501, 1999a.
Masutani et al., Nature 399: 700-704, 1999b.
Matsuda et al., Nature, 404:1011-1013, 2000.
Mayer et al., Biochimica et Biophysica Acta, vol. 858, pp. 161-168, 1986.
Mayhew et al., Biochimica et Biophysica Acta, vol. 775, pp. 169-174, 1984.
Mayhew et al., Methods in Enzymology, vol. 149, pp. 64-77, 1987.
McConnell et al., Biochemistry 35, 8268-8274, 1996.
McDonald et al., Genetics 147: 1557-1568, 1997.
McDonald et al., Genomics 60: 20-30, 1999.
McDonald et al., Nat. Genet. 15, 417-474, 1997
McNeall et al., Gene, 76:81, 1989.
Merrifield, J. Am. Chem. Soc. 85:2149 (1963).
Michel and Westhof, J. Mol. Biol., 216:585-610, 1990.
Miksicek et al., Cell, 46:203, 1986.
Miller, et al, Bioorg. Med. Chem. Lett. 4:2657 (1994).
Mitchell et al., Ann. N.Y. Acad. Sci., 690:153-166, 1993.
Mitchell et al., J Clin Oncol., 8(5):856-859, 1990.
Moon, et al., Tetrahedron Lett. 35:8915 (1994).
Moore, et al., J. Cell Biol. 147:471-80 (1999).
Mordacq and Linzer, Genes and Dev., 3:760, 1989.
Moreau et al., Nucl. Acids Res., 9:6047, 1981.
Mori et al., Cancer Res., 54:3396-3397, 1994.
Morton and Ravindranath, M. H. Current concepts concerning melanoma vaccines. In Tumor Immunology, Dalgleish AG (ed.), London: Cambridge University Press, 1-55, 1996.
Morton et al., Ann. Surg. 216: 463-482, 1992.
Muesing et al., Cell, 48:691, 1987.
Nakamura et al., In: Enzyme Immunoassays: Heterogeneous and Homogeneous Systems, Chapter 27, 1987.
Needels, et al., Proc. Natl. Acad. Sci. U.S.A. 90:10700 (1993).
Nelson et al., Nature 382, 729-731, 1996.
Nestler, et al., J. Org. Chem. 59:4723 (1994).
Neugebauer, et al., Genes & Dev. 11: 1148 (1997).
Ng et al., Nuc. AcidsRes., 17:601, 1989.
Nicolas and Rubenstein, In: Vectors: A survey of molecular cloning vectors and their uses, Rodriguez and Denhardt (eds.), Stoneham: Butterworth, pp. 493-513, 1988.
Nicolau and Sene, Biochim. Biophys. Acta, 721:185-190, 1982.
Nicolau et al., Methods Enzymol., 149:157-176, 1987.
Nielsen, et al., J. Am. Chem. Soc. 115:9812 (1993).
Nikolaiev, et al., Peptide Res. 6:161 (1993).
Nobori et al., Nature, 368:753-756, 1995.
Nomoto et al., Gene, 236(2):259-271, 1999.
Ogi et al., Genes Cells, 4:607-618, 2000.
Ohara et al., Proc. Nat'l Acad. Sci. USA, 86: 5673-5677, 1989.
Ohashi et al., Genes Dev., in press, 2000.
Ohashi et al., Genes Dev., in press, 2000b.
Ohlmeyer, et al., Proc. Natl. Acad. Sci. USA. 90:10922 (1993).
Ohmori et al., Mut. Res. 347: 1-7, 1995.
Okamoto et al., Proc. Nat'l Acad Sci. USA, 91:11045-11049, 1994.
Omirulleh et al., Plant Mol. Biol., 21:415-28, 1993.
Ondek et al., EMBO J., 6:1017, 1987.
Orlow et al., Cancer Res., 54:2848-2851, 1994.
Ornitz et al, Mol. Cell. Biol., 7:3466, 1987.
Osheroff et al., J. Biol. Chem., 274:20749-20752, 1999.
Osheroff et al., J. Biol. Chem., 274:3642-3650, 1999.
Ostresh, et al, Proc. Natl. Acad. Sci. U.S.A. 91:11138 (1994).
Owens, R. A., et al, Biochem. Biophys. Res. Commun. 181:402 (1991).
Palmiter et al., Nature, 300:611, 1982.
Palukaitis et al., Virology, 99:145-151, 1979.
Patek, et al., Tetrahedron Lett 35:9169 (1994).
Patek, et al., Tetrahedron Lett. 36:2227 (1995).
Pavia, M. R., et al, Bioorg. Med. Chem. Lett. 3:387 (1993).
Pease et al., Proc. Natl. Acad. Sci. USA, 91:5022-5026, 1994.
Pech et al., Mol. Cell. Biol., 9:396, 1989.
Pelletier and Sonenberg, Nature, 334:320-325, 1988.
Perez-Stable and Constantini, Mol. Cell. Biol., 10: 1116, 1990.
Perriman. et al., Gene, 113:157-163, 1992.
Perrotta and Been, Biochemistry 31:16, 1992.
Picard and Schaffner, Nature, 307:83, 1984.
Pietras et al, Oncogene, 17(17):2235-49, 1998.
Pinkert et al., Genes and Dev., 1:268, 1987.
Pirrung, et al., J. Am. Chem. Soc. 117:1240 (1995).
Poch et al., EMBO J. 8, 3867-3874, 1989.
Ponta et al., Proc. Nat'l Acad. Sci. USA., 82:1020, 1985.
Popperl, et al., Mol. Cell. 6:255-67, 2000.
Porton et al., Mol. Cell. Biol., 10:1076, 1990.
Potrykus et al., Mol. Gen. Genet., 199:183-188, 1985.
Prelich et al., Nature 326, 517-520, 1987.
Prody, G. A. et al., Science, 231, 1577-1580, 1986.
Qin et al., Proc. Nat'l Acad Sci. USA, 95(24):1411-6, 1998.
Queen and Baltimore, Cell, 35:741, 1983.
Quinn et al., Mol. Cell. Biol., 9:4713, 1989.
Radman, Nature 401: 866-869, 1999.
Rajewsky et al., J. Clin. Invest. 98: 600-603, 1996.
Rajewsky, Nature 381: 751-758, 1996.
Ramirez-Solis et al., Methods Enzymol. 225: 855-879m 1993.
Rano, et al., Tetrahedron Lett. 36:37879 (1995).
Ravindranath and Morton, Intern. Rev. Immunol. 7: 303-329, 1991.
Redondo et al., Science, 247:1225, 1990.
Reinhold-Hurek and Shub, Nature, 357:173-176, 1992.
Reisman and Rotter, Mol. Cell. Biol., 9:3571, 1989.
Remington's Pharmaceutical Sciences, 15^{th} ed., pages 1035-1038 and 1570-1580, Mack Publishing Company, Easton, Pa., 1980.
Resendez Jr. et al., Mol. Cell. Biol., 8:4579, 1988.
Reuven et al., J. Biol. Chem. 274: 31763-31766, 1999.
Reuven et al., Mol Cell 2: 191-199, 1998.
Richter et al., Mol. Gen. Genet. 231, 194-200, 1992.
Ridgeway, In: Vectors: A survey of molecular cloning vectors and their uses, Rodriguez R L, Denhardt D T, ed., Stoneham:Butterworth, pp. 467-492, 1988.
Ripe et al., Mol. Cell. Biol., 9:2224, 1989.
Rippe et al., Mol. Cell Biol., 10:689-695, 1990.
Rittling et al., Nucl. Acids Res., 17:1619, 1989.
Roest et al., Cell, 86:799-810, 1996.
Rosen et al., Cell, 41:813, 1988.
Rosenberg et al., Ann. Surg., 210:474, 1989.
Rosenberg et al., N. Engl. J. Med., 319:1676, 1988.
Roush et al., Mol. Gen. Gen. 257, 686-692, 1998.
Sachs, Cell 74, 413-421, 1993.
Saitou et al., Mol. Biol. Evol. 4, 406-425, 1987.
Sakai and Miyazaki Biochem. Biophys. Res. Commun. 237: 318-324, 1997.
Sakai et al., Genes and Dev., 2:1144, 1988.
Salmon, S. E., et al, Proc. Natl. Acad. Sci. U.S.A. 90:11708 (1993).
Sambrook et al., In: Molecular Cloning: A Laboratory Manual, 2nd Ed., Cold Spring Harbor Press, Cold Spring Harbor, N.Y., 1989.
Sarver, et al, Science, 247:1222-1225, 1990.
Satake et al., J Virology, 62:970, 1988.
Saturno et al., J. Mol. Biol. 283, 633-642, 1998.
Scanlon et al., Proc. Nat'l Acad. Sci. USA, 88:10591-10595, 1991.
Schaffner et al., J. Mol. Biol., 201:81, 1988.
Schmidt, et al., Bioorg. Med. Chem. Lett. 3:441 (1993).
Scott, et al, Science, 249:386 (1990).
Searle et al., Mol. Cell. Biol., 5:1480, 1985.
Semenza, Cell, 98:281 (1999).
Semenza, Annu. Rev. Cell Dev. Biol., 15:551 (1999).
Semenza, Genes Dev., 14:1983 (2000).
Serrano et al., Nature, 366:704-707, 1993.
Serrano et al., Science, 267:249-252, 1995.
Sharp and Marciniak, Cell, 59:229, 1989.
Shaul and Ben-Levy, EMBO J., 6:1913, 1987.
Sherman et al, Mol. Cell. Biol., 9:50, 1989.
Shinoda, K. et al., Colloidal Surfactant, Academic Press, especially “The Formation of Micelles”, Ch. 1, 1-96, 1963.
Shoemaker et al., Nature Genetics, 14:450-456, 1996.
Simon, et al, Proc. Natl. Acad. Sci. U.S.A. 89:9367 (1992).
Simon, et al., Cancer Res. 60:328 (2000).
Sioud et al., J. Mol. Biol., 223:831-835, 1992.
Sleigh and Lockett, J. EMBO, 4:3831, 1985.
Smith, et al., Bioorg. Med. Chem. Lett. 4:2821 (1994).
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Storey, Am. J. Physiol. 258:R559-68, 1990).
Storey, Comp. Biochem. Physiol A Physiol, 117:319-26, 1997).
Storey, in: Surviving hypoxia mechanisms of control and adaptation (ed. Hochachka, et al), pp. 253-269 (1993).
Stuart et al, Nature, 317:828, 1985.
Sullivan and Peterlin, Mol. Cell. Biol., 7:3315, 1987.
Swartzendruber and Lehman, J. Cell. Physiology, 85:179, 1975.
Symons, Ann. Rev. Biochem., 61:641-671, 1992.
Symons, Nucl. Acids Res., 9:6527-6537, 1981.
Szoka et al., Proc. Natl. Acad. Sci., 75:4194-4198, 1978.
Takebe et al., Mol. Cell. Biol, 8:466, 1988.
Tang et al., Proc. Natl. Acad. Sci. USA 95: 9755-9760, 1998.
Tang et al., Nature 404: 1014-1018, 2000.
Tang et al., Proc. Natl. Acad. Sci. USA 96: 8919-8924, 1999.
Tavernier et al, Nature, 301:634, 1983.
Taylor and Kingston, Mol. Cell. Biol., 10:165, 1990a.
Taylor and Kingston, Mol. Cell. Biol., 10:176, 1990b.
Taylor et al, J. Biol. Chem., 264:15160, 1989.
Temin, In: Gene Transfer, Kucherlapati (ed.), New York: Plenum Press, pp. 149-188, 1986.
Templeton et al., Nat. Biotechnol., 15(7):647-52, 1997.
Terrett, N. K., et al, Tetrahedron, 51:8135 (1995).
Terrett, N. K., et al, Bioorg. Med. Chem. Lett. 5:917 (1995).
Thiesen et al, J. Virology, 62:614, 1988.
Thomas et al, Biochemistry, 30:11751-11759, 1991.
Thompson et al. Nature Medicine, 1:277-278, 1995.
Thompson, et al., Nucleic Acids Res. 22, 4673-4680, 1994.
Timmons, et al., Nature 395(6705):854 (1998).
Tonk et al., Amer. Jour. Med. Genet. 61, 16-20, 1996.
Treisman, Cell, 42:889, 1985.
Tronche et al., Mol. Biol. Med., 7:173, 1990.
Tronche et al., Mol. Cell. Biol., 9:4759, 1989.
Trudel and Constantini, Genes and Dev., 6:954, 1987.
Tsujimoto and Croce, Proc. Natl. Acad. Sci. USA, 83(14):5214-8, 1986.
Tsujimoto et al., Science, 228(4706): 1440-3, 1985.
Tsumaki et al., J. Biol. Chem. 273(36):22861-22864, 1998.
Tyndall et al., Nuc. Acids. Res., 9:6231, 1981.
Vannice and Levinson, J. Virology, 62:1305, 1988.
Van Voorhies, et al., J. Exp. Biol. 203 pt. 16:2467 (2000).
Vasseur et al., Proc. Nat'l Acad Sci. USA., 77:1068, 1980.
Virgilio, et al., J. Am. Chem. Soc. 116:11580 (1994).
Wada et al., Nucleic Acids Res., 18:2367-2411, 1990.
Wagner et al., Mol. Cell. 40: 281-286, 1999.
Walker et al, Proc. Nat'l Acad Sci. USA, 89:392-396 1992.
Wang and Calame, Cell, 47:241, 1986.
Wang et al., In Eukaryotic DNA Replication. A Practical Approach (ed. S. Cotterill), pp. 67-92. Oxford University Press, NY, 1999.
Wang et al., Science, 289:774-9, 2000.
Wawrzynczak & Thorpe, Cancer Treat. Res., 37:239-51, 1988.
Weber et al., Cell, 36:983, 1984.
Weinberg, Science, 254:1138-1145, 1991.
Weinberger et al Mol. Cell. Biol., 8:988, 1984.
Westerfield, The zebrafish book. Guide for the laboratory use of zebrafish (Univ. of Oregon Press, Eugene), 1995.
Wingrove, et al., Cell 98:105 (1999).
Winoto and Baltimore, Cell, 59:649, 1989.
Wolf et al., Comput. Appl. Biosci., 4(1):187-191, 1988.
Wong et al., Gene, 10:87-94, 1980.
Woodgate & Sedgwick, Mol. Microbiol. 6, 2213-2218., 1992
Wu et al., Biochem. Biophys. Res. Commun., 233(1):221-6, 1997.
Yager, et al., Biochem. Cell Biol. 75:535-50, 1997.
Yuan and Altman, Science, 263:1269-1273, 1994.
Yuan et al., Proc. Nat'l Acad. Sci. USA, 89:8006-8010, 1992.
Yutzey et al. Mol. Cell. Biol., 9:1397, 1989.
Zamir, et al., Mol. Cell. Biol. 17:529-36, 1997.
Zaychikov et al., Science 273, 107-109, 1996.
Zhao-Emonet et al., Biochem. Biophys. Acta., 1442(2-3): 109-19, 1998.
Zipperlen, et al., A Comprehensive Genome-Wide RNAi Screen in C. Elegans (asbtract), 2000 European Worm Meeting.
Zuckerman, et al, J. Am. Chem. Soc. 114:10646 (1992).
Zuckerman, et al, J. Med. Chem. 37:2678 (1994).

What is claimed is:

1. A method for cryopreserving biological material comprising:
a) first incubating the biological material under anoxic conditions for an effective amount of time for the biological material to enter stasis; and
b) then cryopreserving the biological material.

2. The method of claim 1, wherein the biological material is a cell, tissue, or organism.

3. The method of claim 2, wherein the biological material is a cell.

4. The method of claim 3, wherein the cell is a sex cell.

5. The method of claim 3, wherein the cell is comprised in an embryo.

6. The method of claim 5, wherein the embryo is a vertebrate embryo.

7. The method of claim 6, wherein the vertebrate embryo is mammalian.

8. The method of claim 2, wherein the biological material is tissue.

9. The method of claim 8, wherein the tissue is an organ.

10. The method of claim 2, wherein the biological material is an organism.

11. The method of claim 10, wherein the organism is an invertebrate.

12. The method of claim 10, wherein the organism is a vertebrate.

13. The method of claim 1, wherein the biological material is incubated under anoxic conditions for more than 30 minutes.

14. The method of claim 13, wherein the biological material is incubated under anoxic conditions for more than 1 hour.

15. The method of claim 13, wherein the biological material is incubated under anoxic conditions for more than 2 hours.

16. The method of claim 1, wherein cryopreserving the biological material comprises perfusing the biological material with a cryoprotectant and lowering the temperature of the biological material.

17. The method of claim 16, wherein the temperature is lowered to below 0° C.

18. A method for preserving biological material comprising:
a) incubating the biological material under hypoxic conditions for an effective amount of time for the biological material to enter stasis.

19. The method of claim 18, further comprising lowering the temperature of the biological material.

20. The method of claim 18, further comprising incubating the biological material under normoxic conditions to reverse stasis.

21. The method of claim 18, wherein the biological material is incubated under conditions of less than 10% oxygen.

22. The method of claim 21, wherein the biological material is incubated under conditions of less than 5% oxygen.

23. The method of claim 18, wherein the biological material exhibits signs of trauma.

24. The method of claim 19, wherein the temperature is lowered to below 15° C.

25. The method of claim 24, wherein the temperature is lowered to below 10° C.

26. The method of claim 18, wherein the biological material is an organism.

27. The method of claim 18, wherein the organism is a vertebrate.

28. The method of claim 27, wherein the vertebrate is a Danio rerio embryo.

29. A method for screening for an antitumor compound comprising:
a) incubating a first anoxia-resistant organism under hypoxic conditions sufficient to permit the organism to enter stasis;
b) incubating the first organism with a candidate compound;
c) observing the first organism for viability; and
d) comparing the first organism's viability against a second anoxia-resistant organism's viability incubated under normoxic conditions in the presence of the candidate compound,
wherein viability of the second organism and lack of viability of the first organism identifies the candidate compound as an anti-tumor compound.

30. The method of claim 29, wherein the hypoxic conditions have less than 10% oxygen.

31. The method of claim 30, wherein the hypoxic conditions are anoxic.

32. The method of claim 29, further comprising removing the candidate compound from the first and second organisms.

33. The method of claim 29, wherein observing the first organism for viability comprises observing them for movement.

34. The method of claim 29, wherein the first and second anoxia-resistant organisms are nematodes.

35. The method of claim 34, wherein the nematode is Caenorabditis elegans.

36. The method of claim 29, wherein the first and second anoxia-resistant organisms are vertebrate organisms.

37. The method of claim 36, wherein the vertebrate organisms are embryos.

38. The method of claim 37, wherein the embryos are Danio rerio.

39. The method of claim 29, wherein the organisms are in a hypoxic environment for at least 30 minutes.

40. The method of claim 39, wherein the organisms are in a hypoxic environment for at least 1 hour.

41. The method of claim 40, wherein the organisms are in a hypoxic environment for at least 2 hours.

42. An antitumor composition comprising an antitumor compound identified by a process comprising:
a) incubating a first anoxia-resistant organism under hypoxic conditions sufficient to induce stasis;
b) incubating the first organism with a candidate compound; and
c) comparing the first organism's viability against a second anoxia-resistant organism's viability incubated under normoxic conditions in the presence of the candidate compound,
wherein the compound is an antitumor compound if the first anoxia-resistant organism is no longer viable and the second anoxia-resistant organism is viable after incubation with the candidate compound.

43. The composition of claim 42, wherein the first and second anoxia-resistant organisms are nematodes.

44. The composition of claim 42, wherein the first and second anoxia-resistant organisms are embryos.

45. The composition of claim 44, wherein the first and second anoxia-resistant organisms are vertebrate embryos.

46. The composition of claim 42, wherein the hypoxic conditions are less than 10% oxygen.

47. The composition of claim 46, wherein the hypoxic conditions are anoxic.

48. The composition of claim 47, wherein the first organism is incubated under hypoxic conditions for more than 30 minutes.

49. The composition of claim 48, wherein the first organism is incubated under hypoxic conditions for more than 1 hour.

50. The composition of claim 42, wherein the candidate compound is a small molecule.

51. The composition of claim 42, wherein the anti-tumor compound comprises mitotracker red.

52. A method for killing cancer cells in a patient with a tumor comprising administering to the patient an therapeutically effective amount of an antitumor compound identified by the process comprising:
a) incubating a first anoxia-resistant organism under hypoxic conditions sufficient to permit the organism to enter stasis;
b) incubating the first organism with a candidate compound;
c) observing the first organism for viability; and
d) comparing the first organism's viability against a second anoxia-resistant organism's viability incubated under normoxic conditions in the presence of the candidate compound,
wherein viability of the second organism and lack of viability of the first organism identifies the candidate compound as an anti-tumor compound.

53. The method of claim 52, wherein the compound is administered directly to the tumor.

54. The method of claim 53, wherein the compound is injected into the tumor.

55. The method of claim 52, further comprising administering to the patient chemotherapy, radiotherapy, immunotherapy, or gene therapy.

56. The method of claim 52, further comprising resecting at least a portion of the tumor from the patient.

57. The method of claim 52, wherein the compound is mitotracker red.

58. A method for screening for a compound that induces stasis in biological material comprising:
a) incubating a first organism capable of stasis with a candidate compound; and
b) evaluating the first organism for stasis,
wherein the compound is a stasis inducer if the first organism exhibits stasis after exposure to the compound.

59. The method of claim 58, further comprising
c) comparing the ability to enter stasis in the first organism with a second organism not incubated or no longer incubated with the candidate compound.

60. The method of claim 58, further comprising
d) removing the compound from the first organism; and
e) evaluating the first organism for loss of stasis,
wherein the compound is a reversible stasis inducer if the first organism exhibits stasis after incubation with the compound, but no longer exhibits stasis after the compound is removed.

61. A method of screening for a compound that improves the ability to undergo stasis comprising:
a) incubating a first organism capable of undergoing stasis under hypoxic conditions;
b) exposing the first organism to a candidate compound;
c) incubating a second organism capable of undergoing stasis under the same hypoxic conditions as the first organism;
d) comparing the first organism and the second organism.

62. A stasis inducer compound identified by a process comprising:
a) incubating an organism capable of entering stasis with a candidate compound; and
b) evaluating the organism for stasis,
wherein the compound is a stasis inducer if the organism exhibits stasis after incubation with the compound.

63. A reversible stasis inducer compound identified by a process comprising:
a) incubating an organism capable of entering stasis with a candidate compound;
b) comparing the organism with an organism capable of entering stasis that is no longer incubated with the candidate compound,
wherein the compound is a stasis inducer if the organism exhibits stasis after incubation with the compound, but does not exhibit stasis when no longer incubated with the compound.

64. The reversible stasis inducer compound of claim 63, wherein the organism no longer exposed to the candidate compound in c) is the organism of a).

65. A method of inducing stasis in biological material comprising administering to the biological material a stasis inducer compound.

66. The method of claim 65, further comprising lowering the temperature of the biological material.

67. The method of claim 65, wherein the biological material is an organ.

68. The method of claim 65, wherein the biological material is tissue.

69. The method of claim 65, wherein the biological material is an organism.

70. The method of claim 69, wherein the organism is a vertebrate organism.

71. The method of claim 70, wherein the vertebrate organism is an embryo.

72. The method of claim 70, wherein the vertebrate organism is a mammal.

73. The method of claim 72, wherein the mammal is a human.

74. A method of inducing stasis in a cell of an organism comprising administering to the cell of the organism an effective amount of a stasis inducer compound identified by a process comprising:
a) incubating an organism capable of entering stasis with a candidate compound;
b) evaluating the organism for loss of stasis after the compound is removed,
wherein the compound is a stasis inducer if the organism exhibits stasis after incubation with the compound, but no longer exhibits stasis after the compound is removed.

75. A method of inducing stasis in an organism comprising administering to the organism an effective amount of a stasis inducer compound identified by a process comprising:
a) incubating an organism capable of entering stasis with a candidate compound;
b) evaluating the organism for loss of stasis after the compound is removed,
wherein the compound is a stasis inducer if the organism exhibits stasis after incubation with the compound, but no longer exhibits stasis after the compound is removed.

76. A method of identifying a modulator of stasis in a biological material undergoing stasis comprising:
a) incubating a first biological material undergoing stasis with a candidate compound, wherein the first biological material is incubated under hypoxic conditions;
b) evaluating the first biological material for loss of stasis;
c) comparing the first biological material with a second biological material undergoing stasis but not incubated with the candidate compound, wherein a difference in stasis between the first and second biological materials identifies the compound as a candidate modulator of stasis.
